(19) United States
(12) Patent Application Publication
(10) Pub. No.: US 2015/0185238 A1
(43) Pub. Date: Jul. 2, 2015

(54) NOVEL METHOD FOR THE DIAGNOSIS OF ENDOMETRIOSIS

(71) Applicants: HELMHOLTZ ZENTRUM MUNCHEN - DEUTSCHES FORSCHUNGSZENTRUM FUR GESUNDHEIT UND UMWELT; (GMBH), Neuherberg (DE); UNIVERZA V LJUBLJANI, Ljubjana (SI)

(72) Inventors: Katja Vouk, Hohenkirchen-Siegertsbrunn (DE); Tea Lanisnik-Rizner, Domzale (SI); Jerzy Adamski, Munchen (DE); Cornelia Prehn, Munchen (DE); Martina Ribic-Pucelj, Bled (SI)

(73) Assignee: HELMHOLTZ ZENTRUM MUNCHEN, Neuherberg (DE)

(21) Appl. No.: 14/404,887

(22) PCT Filed: May 31, 2013
(86) PCT No.: PCT/EP2013/061284

§ 371 Date: Dec. 1, 2014

(30) Foreign Application Priority Data
May 31, 2012 (EP) 12170213.8

(51) Int. Cl. G01N 33/92; G01N 33/68

(52) U.S. Cl. CPC G01N 33/92; G01N 33/6812; G01N 33/689; G01N 2800/364; G01N 2458/15; G01N 2570/00

(57) ABSTRACT

The present invention relates to a method of diagnosing endometriosis, the method comprising (i) determining in a sample obtained from a subject: (a) the concentration of SMOH C16:1 and the ratio of the concentration of PCaa C36:2 to the concentration of PCae C34:2; (b) the concentration of PCae C30.0, the ratio of the concentration of PCaa C36.2 to the concentration of PCae C36.2 and the ratio of the concentration of Trp to the concentration of PCae C34.0; (c) the concentration of PCae C36.1, the ratio of the concentration of PCaa C36.2 to the concentration of PCae C36.2 and the ratio of the concentration of Trp to the concentration of PCae C34.0; (d) the concentration of SM C16.1, the ratio of the concentration of PCaa C36.2 to the concentration of PCae C36.2 and the ratio of the concentration of Trp to the concentration of PCae C34.0; (e) the concentration of SM C16.1, the ratio of the concentration of PCaa C36.2 to the concentration of PCae C36.2 and the ratio of the concentration of Arg to the concentration of PCae C34.2; (f) the concentration of SM C16:1, the ratio of the concentration of PCaa C36:2 to the concentration of PCae C36:2 and the ratio of the concentration of Trp to the concentration of PCae C34:2; and/or (g) the concentration of SMOH C22:2, the ratio of the concentration of PCaa C36:2 to the concentration of PCae C36:2 and the ratio of the concentration of Trp to the concentration of PCae C34:0 and (ii) comparing the values determined in (i) with values obtained from healthy subjects; wherein an increase in the concentration of the single metabolites in combination with a decrease in the ratio(s) as compared to values obtained from healthy subjects is indicative of endometriosis. The invention further relates to a kit comprising metabolite standards labelled with (a) stable isotope(s) or chemically similar compounds not naturally occurring in the human sample.

The present invention relates to a method of diagnosing endometriosis, the method comprising (i) determining in a sample obtained from a subject: (a) the concentration of SMOH C16:1 and the ratio of the concentration of PCaa C36:2 to the concentration of PCae C34:2; (b) the concentration of PCae C30:0, the ratio of the concentration of PCaa C36:2 to the concentration of PCae C36:2 and the ratio of the concentration of Trp to the concentration of PCae C34:0; (c) the concentration of PCae C36:1, the ratio of the concentration of PCaa C36:2 to the concentration of PCae C36:2 and the ratio of the concentration of Trp to the concentration of PCae C34:0; (d) the concentration of SM C16:1, the ratio of the concentration of PCaa C36:2 to the concentration of PCae C36:2 and the ratio of the concentration of Trp to the concentration of PCae C34:0; (e) the concentration of SM C16:1, the ratio of the concentration of PCaa C36:2 to the concentration of PCae C36:2 and the ratio of the concentration of Arg to the concentration of PCae C34:2; (f) the concentration of SM C16:1, the ratio of the concentration of PCaa C36:2 to the concentration of PCae C36:2 and the ratio of the concentration of Trp to the concentration of PCae C34:2; and/or (g) the concentration of SMOH C22:2, the ratio of the concentration of PCaa C36:2 to the concentration of PCae C36:2 and the ratio of the concentration of Trp to the concentration of PCae C34:0 and (ii) comparing the values determined in (i) with values obtained from healthy subjects; wherein an increase in the concentration of the single metabolites in combination with a decrease in the ratio(s) as compared to values obtained from healthy subjects is indicative of endometriosis. The invention further relates to a kit comprising metabolite standards labelled with (a) stable isotope(s) or chemically similar compounds not naturally occurring in the human sample.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Endometriosis is a gynecological medical condition in which cells from the lining of the uterus, the endometrium, are found growing outside the uterine cavity, the pelvic peritoneum, different parts of the rectovaginal tract, and most commonly on the ovaries. This thus forms three different entities that show different pathogenesis: ovarian endometriosis, peritoneal endometriosis, and deep infiltrating endometriosis, respectively (Guidice and Kao, 2004). A major symptom of endometriosis is recurring pelvic pain. Endometriosis lesions may also react to hormonal stimulation and cause “bleeding” at the time of menstruation. The blood can accumulate and cause swelling as well as triggering inflammatory responses with the activation of cytokines, which in turn may cause pain. The pain experienced may also be the result of the binding of internal organs to each other via internal scar tissue, thereby causing organ dislocation. Fallopian tubes, ovaries, the uterus, the bowels, and the bladder can for example become bound together in a painful manner. Endometriosis has been reported to be associated with infertility, which may be related to scar formation or to anatomical alterations; however, it is possible that endometriosis interferes by releasing cytokines and other chemical agents that interfere with reproduction.

Endometriosis affects an estimated 176 million women worldwide and is one of the most common diseases, more frequent than cancer and diabetes. The nature of this disease is heterogeneous and comprises ovarian, peritoneal as well as deep infiltrating endometriosis. While the estimated prevalence is 6 to 10% in the general female population, the frequency is 35 to 50% in women with pain, infertility, or both (Giudice and Kao, 2004). The disease may stay asymptomatic for a longer period of time until it becomes overt and advanced. Establishing a correct diagnosis of endometriosis is often problematic, because the symptoms are non-specific and associated with a number of different conditions (Giudice and Kao, 2004). The accepted standard for diagnosis of endometriosis is surgical visual inspection of the pelvic organs, an invasive procedure that requires an experienced surgeon, general anesthesia and includes surgery associated risks. As a result of this, it can take up to 12 years before affected women obtain a diagnosis and receive appropriate treatment (Hadfield et al., 1996). A therapy in the earlier stage of disease would reduce life discomfort, infertility and would decrease a risk of inflammation or other complications. An accurate blood test could avoid the need for an invasive procedure (Brosens et al. 2003) thus enabling earlier diagnosis and treatment (May et al., 2010).

Although over 100 potential biomarkers of endometriosis, including cytokines, antibodies, specific cell populations, components of the complement pathway and soluble HLA molecules, serum glycoproteins, cell adhesion molecules, growth factors, hormones, proteins detected by proteomics tools, pro-angiogenic factors, apoptotic factors, and a number of other proteins, were reported (reviewed in May et al., 2010), not a single one, nor a panel of biomarkers have yet unequivocally been shown as clinically useful (May et al., 2010).

Current diagnostic methods such as trans-vaginal ultrasound, magnetic resonance and biomarker CA125 lack sufficient sensitivity and specificity. The CA125 biomarker has been used in clinical practice for over 20 years. However, a meta-analysis published in 1998 (Mol et al. 1998) showed that the biomarker's performance in diagnosing endometriosis was low, even though it showed some promise in detecting more severe disease (May et al., 2010).

Mihalyi et al. 2010 describes a combination of six plasma biomarkers for use in noninvasive diagnosis of minimal to mild endometriosis. The authors found that these biomarkers provide a high sensitivity (87-92%) and an acceptable specificity (60-71%) during the secretory phase and the menstrual phase. Although this combination of biomarkers reached very high sensitivity, it still lacks specificity.

WO 2010/107734 describes a method of detecting endometriosis in a mammalian subject based on levels of sphingomyelin or phosphatidylcholine, or combinations thereof. A diagnosis of the subject having endometriosis is given when the measured level of sphingomyelin or of phosphatidylcholine is decreased as compared to a predetermined level obtained from a healthy subject. By employing a Plasma SM Assay for the measurement of sphingomyelin and an enzymatic measurement of plasma levels of phosphatidylcholine, only the overall chemical classes “sphingomyelin” or “phosphatidylcholine” can be determined, which generally include hundreds of different molecules. Such an approach of solely measuring a chemical class instead of specific, individual molecules is not selective enough as demonstrated by the studies in which only distinct lipids were indicative for a phenotype whereas the whole chemical class was not (Griffin J L, Atherton H, Shockcor J, Atzori L (2011) Metabolomics as a tool for cardiac research. Nature reviews Cardiology 8: 630-643, Griffiths W J, Koal T, Wang Y, Kohl M, Enot D P, Deigner H P (2010) Targeted metabolomics for biomarker discovery. Angew Chem Int Ed Engl 49: 5426-5445, Spickett C M, Wiswedel I, Siems W, Zarkovic K, Zarkovic N (2010) Advances in methods for the determination of biologically relevant lipid peroxidation products. Free radical research 44: 1172-1202, Zhang L, Jia X, Peng X, Ou Q, Zhang Z, Qiu C, Yao Y, Shen F, Yang H, Ma F, Wang J, Yuan Z (2010) Development and validation of a liquid chromatography-mass spectrometry metabonomic platform in human plasma of liver failure caused by hepatitis B virus. Acta biochimica et biophysica Sinica 42: 688-698).

As mentioned above, all current noninvasive diagnostic methods for endometriosis lack sensitivity and specificity. Thus, although a large amount of effort is directed at identifying noninvasive methods for diagnosing endometriosis, there is still a need to provide noninvasive methods that enable the early detection of endometriosis with an increased specificity of detection.

This need is addressed by the provision of the embodiments characterised in the claims.

Accordingly, the present invention relates to a methods of diagnosing endometriosis, the method comprising (i) determining in a sample obtained from a subject: (a) the concentration of SMOH C16:1 and the ratio of the concentration of PCaa C36:2 to the concentration of PCae C34:2; (b) the concentration of PCae C30:0, the ratio of the concentration of PCaa C36.2 to the concentration of PCae C36:2 and the ratio of the concentration of Trp to the concentration of PCae C34.0; (c) the concentration of PCae C36:1, the ratio of the concentration of PCaa C36:2 to the concentration of PCae C36:2 and the ratio of the concentration of Trp to the concentration of PCae C34:0; (d) the concentration of SM C16:1, the ratio of the concentration of PCaa C36:2 to the concentration of PCae C36:2 and the ratio of the concentration of Trp to the concentration of PCae C34:0; (e) the concentration of SM C16:1, the ratio of the concentration of PCaa C36:2 to the concentration of PCae C36:2 and the ratio of the concentration of Arg to the concentration of PCae C34:2; (f) the concentration of SM C16:1, the ratio of the concentration of PCaa C36:2 to the concentration of PCae C36:2 and the ratio of the concentration of Trp to the concentration of PCae C34:2; and/or (g) the concentration of SMOH C22:2, the ratio of the concentration of PCaa C36:2 to the concentration of PCae C36:2 and the ratio of the concentration of Trp to the concentration of PCae C34:0 and (ii) comparing the values determined in (i) with values obtained from healthy subjects; wherein an increase in the concentration of the single metabolites in combination with a decrease in the ratio(s) as compared to values obtained from healthy subjects is indicative of endometriosis.

In an alternative embodiment, the method of the present invention relates to a method for producing diagnostically informative concentration values of metabolites for endometriosis, the method comprising the steps recited above.

It is well known in the art that the concentration of a metabolite is the amount of said metabolite in a specified volume of the sample. The ratio of two concentrations is derived by dividing the first concentration by the second concentration, e.g. the ratio of the concentration of PCaa C36:2 to the concentration of PCae C34:2 corresponds to the concentration of PCaa C36:2 divided by the concentration of PCae C34:2, also represented herein as PCaa C36:2/PCae C34:2.

Means and methods for determining the concentration of metabolites in samples, such as e.g. in blood, are well known in the art and are described in more detail herein below.

The metabolites referred to herein are abbreviated using standard abbreviations well known in the art. Accordingly, “PC” abbreviates phosphatidylcholines, “SM” abbreviates sphingomyelines and “CO” abbreviates free carnitine. The term “Cx:y” is used to describe the total number of carbons (x) and the number of double bonds (y) of all chains. Substitutions of side chains with hydroxy-(OH) residue are indicated. Glycerophospholipids are distinguished with respect to the presence of ester (a) and ether (e) bonds in the glycerol moiety, where two letters (aa=diacyl, ae=acyl-alkyl) denote that the two glycerol positions are each bound to a fatty acid residue, while a single letter (a=acyl or e=alkyl) indicates the presence of a single fatty acid residue. For example “PCae C34:1” denotes a glycerophosphatidylcholine with an acyl (a) and an ether (e) side chain, with 34 carbon atoms in both side chains and a single double bond in one of them.

For the metabolites specifically referred to herein, identification numbers are provided from the databases PubChem (Bolton et al., 2008), Lipidmaps (Murphy et al., 2009), HMDB (Wishart et al., 2007, Wishart et al., 2009), CAS (U.S. National Library of Medicine: ChemIDPlus Advanced) and KEGG (Kyoto Encyclopedia of Genes and Genomes, 2011).

Metabolite SMOH C16:1 is a hydroxysphingomyelin having the formula C₃₉H₇₈N₂O₇P and an exact mass of 717.55466 g/mol. SMOH C16:1 is represented in the CAS database as 136795-02-3 and the KEGG database as C00550.

Metabolite SMOH C22:2 is a hydroxysphingomyelin having the formula C45H88N2O₇P and an exact mass of 799.63291 g/mol. SMOH C22:2 is represented in the KEGG database as C00550.

PCaa C36:2 is a phosphatidylcholine of the formula C₄₄H₈₄NO₈P, having an exact mass of 785.59346 g/mol. PCaa C36:2 is represented in the HMDB database as HMDB08135, HMDB08103, HMDB08039, HMDB08559, HMDB08590, HMDB00593, HMDB07979, HMDB08331, HMDB07888, HMDB08011, HMDB08071, HMDB08070, HMDB07920 and HMDB08299. PCaa C36:2 is further represented in the Lipidmaps database as LMGP01010841, LMGP01010764, LMGP01010765, LMGP01010842, LMGP01010766, LMGP01010767, LMGP01010768, LMGP01010769, LMGP01010849, LMGP01010866, LMGP01010848, LMGP01010867, LMGP01010868, LMGP01010869, LMGP01010890, LMGP01010862, LMGP01010891, LMGP01010863, LMGP01010892, LMGP01010864, LMGP01010865, LMGP01010861, LMGP01010860, LMGP01010619, LMGP01010966, LMGP01010836, LMGP01010835, LMGP01010859, LMGP01010857, LMGP01010876, LMGP01010858, LMGP01010855, LMGP01010838, LMGP01010873, LMGP01010856, LMGP01010837, LMGP01010853, LMGP01010854, LMGP01010851, LMGP01010877, LMGP01010852, LMGP01010850, LMGP01010872, LMGP01010871, LMGP01010870, LMGP01010936 and LMGP01010935. It is further represented in the CAS database as 10015-85-7, 4235-95-4, 52088-89-8, 68737-67-7, 19229-69-7 and 27098-24-4 and in the KEGG database as C03889, C00157, C03631, C03873, C04636 and C01282.

The ether-phospholipid PCae C34:2 has the formula C₄₂H₈₂NO₇P and an exact mass of 743.58289 g/mol. PCae C34:2 has the PubChem accession number 6443157 and is represented in the HMDB database as HMDB08126, HMDB11210, HMDB11209, HMDB08028, HMDB11272, HMDB11240, HMDB11305, HMDB07996, HMDB08093, HMDB11151 and HMDB07997; in the Lipidmaps database as LMGP01020040, LMGP01030005, LMGP01030007, LMGP01030006 and LMGP01020039; in the CAS database as 88542-95-4 and in the KEGG database as C00958.

PCae C30:0 is also an ether-phospholipid and has the formula C₃₈H₇₈NO₇P. It has an exact mass of 691.55159 g/mol and is represented in the Lipidmaps database as LMGP01020013 and LMGP01020012 and in the KEGG database as C04598 C05212.

The ether-phospholipid PCae C36:2 has the formula C₄₄H₈₆NO₇P and an exact mass of g/mol 771.61419. It is represented in the HMDB database as HMDB08094, HMDB11274, HMDB08127, HMDB08063, HMDB08062, HMDB11216, HMDB08324, HMDB11307, HMDB11243 and HMDB11242; the Lipidmaps database as LMGP01030013 and the KEGG database as C00958.

The ether-phospholipid PCae C34:0 has the formula C₄₂H₈₆NO₇P and an exact mass of g/mol 747.61419. It is represented in the Lipidmaps database as LMGP01020033, LMGP01020035, LMGP01020087, LMGP01020088, LMGP01020076, LMGP01020134 and LMGP01020086 and in the KEGG database as C04598 and C05212.

PCae C36:1 is also an ether-phospholipid and has the formula C₄₄H₈₈NO₇P. It has an exact mass of 773.62984 g/mol and is represented in the HMDB database as HMDB08291, HMDB08061, HMDB11215 and HMDB11241; the Lipidmaps database as LMGP01020052 and the KEGG database as C00958.

The term “Trp” relates to tryptophan, which is (2S)-2-amino-3-(1H-indol-3-yl) propanoic acid and has the formula C11H12N2O2 as well as an exact mass of 204.23 g/mol.

The term “Arg” refers to arginine, which is 2-Amino-5-guanidinopentanoic acid. Arg has the formula C6H14N4O2 and an exact mass of 174.2 g/mol.

In accordance with the method of the present invention, a hard or soft copy comprising the concentration values determined is optionally prepared. Non-limiting examples of hard copies include print-outs, hand-written information as well as photographs or the data as originally obtained, for example from a mass spectrometer. Non-limiting examples of soft copies include any form of computer files such as the originally obtained data output from the machine performing the measurements (e.g. a mass spectrometer) or from the respective analysis programme or e.g. word or other text software documents containing the values, as well as e.g. screen shots.

It will be appreciated that the values comprised in said hard or soft copy can e.g. be calculated values, for example in the form of numerical values derived from the measurements as well as the original data as obtained. In accordance with the present invention, the option of preparing a hard or soft copy comprising the concentration values determined can be applied to either the method of diagnosing endometriosis of the present invention, or to the method for producing diagnostically informative concentration values of metabolites for endometriosis of the invention.

In accordance with the present invention, the values determined in step (i) are compared with values obtained from healthy subjects.

Subjects are considered as healthy subjects in accordance with the present invention when they do not have endometriosis. Accordingly, it will be appreciated that the term “healthy subject”, in accordance with the present invention, does not require an overall healthy person. Instead, a healthy person in accordance with the present invention is a person not having endometriosis. Whether a woman has endometriosis can be ascertained by the presence of a plurality, such as e.g. at least three, more preferably at least four, such as at least five and most preferably all of the unspecific diagnostic parameters including: normal fertility, no pelvic pain or no pain in lower abdomen before menstruation, no pain with bowel movements, lack of inflammatory biomarkers, lack of extra menstrual bleeding. However, as final and dependable diagnosis of endometriosis depends on laparoscopic examination, which is an invasive operative procedure, it is preferred that the healthy subjects are subjects for which the absence of endometriosis has been confirmed by laparoscopic examination.

Comparison of measured values with values obtained from healthy subjects in order to derive a diagnosis regarding a specific disease is well established in the art and the skilled person is aware of means and methods of carrying out such a comparison.

For example, samples may be taken from a sufficiently large group of healthy subjects, such as for example at least 10, more preferably at least 75 and most preferably at least 100 healthy subjects. The metabolite values obtained from this group, which are also referred to herein as reference values, are then correlated with the absence of endometriosis. It will be appreciated by the skilled person that determining these reference values in healthy subjects may be carried out prior to performing the present method of the invention, such that the determined values may be used as a reference at later times whenever a sample is analysed in accordance with the method of the present invention; or may be determined in parallel each time a sample is analysed in accordance with the method of the present invention. Such reference values may also be determined only once and stored as a standard for all future tests.

Preferably, the reference values are derived from a population having the same racial background as the women to be diagnosed. For example, when employing the method of the present invention in e.g. caucasian women, the reference values should be obtained from healthy caucasian subjects.

For example, using a group of caucasian (i.e. Slovenian) women as shown in the appended examples, lower and upper limits observed in healthy women were as shown in table 1 below.

TABLE 1
Lower and upper limits observed in healthy women

| | Variable In | Lower limit | Upper limit |
|---|---|---|---|
| Variable | transformed | healthy | healthy |
| SMOH C16.1 | no | 1.3 | 3.2 |
| PCae C30.0 | yes | 0.2 | 0.599 |
| PCae C36.1 | no | 3.1 | 8.4 |
| SMC 16.1 | no | 6.9 | 18.8 |
| SMOH C22.2 | no | 3.8 | 11.1 |
| PCaa C36.2/PCae C34.2 | no | 11.466 | 25.8427 |
| PCaa C36.2/PCae C36.2 | yes | 13.5719 | 24.3687 |
| Trp/PCae C34.0 | yes | 38.2483 | 101.5346 |
| Arg/PCae C34.2 | no | 3.5815 | 15.2593 |
| Trp/PCae C34.2 | yes | 3.8062 | 10.4844 |

Accordingly, when employing the method of the present invention in a group of caucasian women, the above defined reference values for healthy subjects may for example be relied upon.

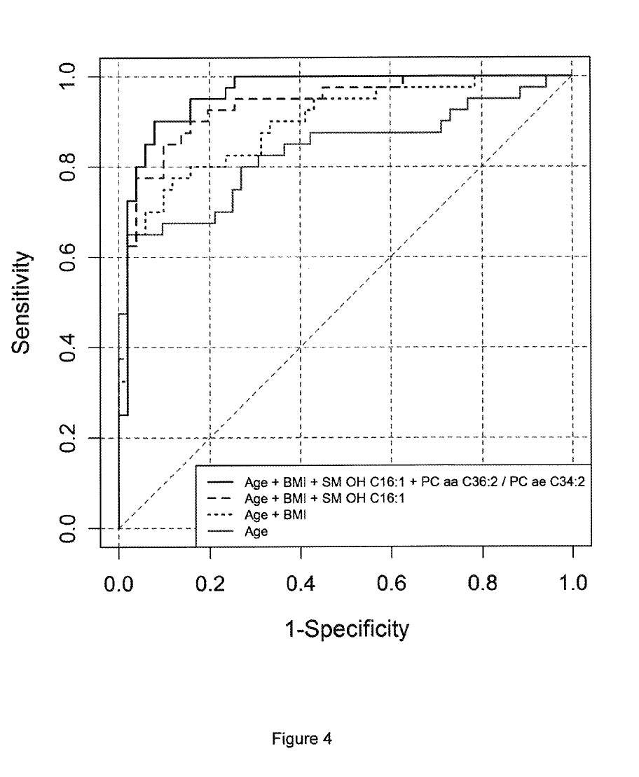

After determination of the above mentioned metabolite values, clinically relevant cut-offs can be derived from the lower and upper limit of values found in healthy subjects. The procedures for calculation of cut-off points are given elsewhere and are fully applicable in accordance with the present invention (Jekel, Katz and Elmore. 2001. Epidemiology, biostatistics and preventive medicine, second edition. W.B. Saunders Company, pp. 106-113.). For example, for each measured test value, sensitivity and specificity are calculate. Sensitivity is a ratio between the number of subjects with a true-positive test result and the total number of diseased subjects. Specificity is a ratio between subjects with a true-negative test result and the total number of subjects without the disease. For deciding about the best cut-off test value a receiver operating characteristic (ROC) curve can be constructed with plotting all the points from calculated pairs of 1-specificity on x-axis and sensitivity on y-axis corresponding to the different test values (see e.g. FIG. 4). The best cut-off test value results from the point closest to the upper left corner of the graph.

For example, based on the data obtained with the population of women analysed in the appended examples, the cut-off points determined for each of the described combinations are as shown in table 2 below.

TABLE 2
Cut-off values as determined based on the population
of women analysed in the appended examples.

| | | | | Metab- | | |
|---|---|---|---|---|---|---|
| | Metab- | | | olite | Ratio1 | Ratio2 |
| | olite | Ratio1 | Ratio2 | Cut-off | Cut-off | Cut-off |
| (a) | SMOH | PCaa C36:2/ | — | 2.4 | 14.6875 | — |
| | C16:1 | PCae C34:2 | | | | |
| (b) | PCae | PCaa C36:2/ | Trp/ | 0.433 | 16.8829 | 63.8349 |
| | C30:0 | PCae C36:2 | PCae C34:0 | | | |
| (c) | PCae | PCaa C36:2/ | Trp/ | 6.9 | 16.8829 | 63.8349 |
| | C36:1 | PCae C36:2 | PCae C34:0 | | | |
| (d) | SM C16:1 | PCaa C36:2/ | Trp/ | 14.8 | 13.5719 | 56.6391 |
| | | PCae C36:2 | PCae C34:0 | | | |
| (e) | SM C16:1 | PCaa C36:2/ | Arg/ | 14.8 | 13.5719 | 6.7921 |
| | | PCae C36:2 | PCae C34:2 | | | |
| (f) | SM C16:1 | PCaa C36:2/ | Trp/ | 12.5 | 16.8829 | 5.1758 |
| | | PCae C36:2 | PCae C34:2 | | | |
| (g) | SMOH | PCaa C36:2/ | Trp/ | 8.2 | 16.8829 | 63.8349 |
| | C22:2 | PCae C36:2 | PCae C34:0 | | | |

The thus determined cut-off points can then serve for comparison when analysing (a) sample(s) obtained from women suspected of having endometriosis.

The approach for predicting the presence of endometriosis in a subject is based on calculating said subject's probability of having endometriosis. The procedure for calculating the probability based on predicting odds of disease from logistic regression equation is described elsewhere and are fully applicable in accordance with the present invention (Bland M 2000. An introduction to medical statistics, third edition, Oxford University Press, pp. 322-323). For example, the logistic regression model is used when the outcome variable is dichotomous (e.g. diseased, nondiseased), whether or not the subject has a particular characteristic (e.g. decreased metabolite concentration, elevated ratio of two metabolites . . . ). The regression model predicts the proportion of individuals that have this characteristic with fitting to the log odds:

Logₑ(p/1−p)=b₀+b₁x₁+b₂x₂+ . . . +bₘxₘ

Where x₁, . . . , xₘ are predictor variables and p is the proportion to be predicted.

The model can then be used for predicting the presence of the outcome (disease) in an investigated subject with putting the measured values of the predictor variable(s) (e.g. decreased metabolite concentration, elevated ratio of two metabolites . . . ) into the equation and calculating the odds of outcome (odds of disease).

For example, the odds of a possible presence of endometriosis (odds of endometriosis) can be calculated as “ln (odds of endometriosis)” from the below stated equation(s) by putting the measured metabolite concentrations, calculated ratios, age and BMI of this subject into the equation(s).

The equations to be used are as follows.

For option (a) employing the metabolite concentration SMOH C16:1 and the ratio of PCaa C36:2 to PCae C34:2 the equation is:

ln(odds of endometriosis)=36.2183+2.2471 (SMOH C16:1)−0.4338 (PCaa C36:2/PCae C34:2)−0.3456 (Age)−6.8785 (ln(BMI)).

For this equation, non-transformed values of the SMOH C16:1 concentration and of the ratio PCaa C36:2 to PCae C34:2 are employed. For options (b) to (e), non-transformed values for the metabolite concentrations for PCae C36:1 and SM C16:1 and for the ratios PCaa C36:2 to PCae C34:2 as well as Arg to PCae C34:2 are employed, while In-transformed values are employed for all other concentrations and ratios. Table 2 summarises this information.

For option (b) employing the metabolite concentration PCae C30:0 and the ratios of PCaa C36:2 to PCae C36:2 and Trp to PCae C34:0 the equation is:

ln(odds of endometriosis)=69.0078+0.7844 (ln(PCae C30:0))−7.2352 (ln(PCaa C36:2/PCae C36:2))−3.8385 (ln(Trp/PCae C34:0))−0.3783 (Age)−5.8349 (ln(BMI)).

For option (c) employing the metabolite concentration PCae C36:1 and the ratios of PCaa C36:2 to PCae C36:2 and Trp to PCae C34:0 the equation is:

ln(odds of endometriosis)=67.32+0.1964 (PCae C36:1)−7.2036 (ln(PCaa C36:2/PCae C36:2))−3.8316 (ln(Trp/PCae C34:0))−0.3938 (Age)−5.802 (ln(BMI)).

For option (d) employing the metabolite concentration SM C16:1 and the ratios of PCaa C36:2 to PCae C36:2 and Trp to PCae C34:0 the equation is:

ln(odds of endometriosis)=67.9235+0.3763 (SM C16.1)−7.2718 (ln(PCaa C36.2/PCae C36.2))−3.6437 (ln(Trp/PC.ae.C34.0))−0.4013 (Age)−7.2908 (ln(BMI)).

For option (e) employing the metabolite concentration SM C16:1 and the ratios of PCaa C36:2 to PCae C36:2 and Arg to PCae C34:2 the equation is:

ln(odds of endometriosis)=62.7129+0.5651 (SM C16:1)−8.7831 (ln(PCaa C36:2/PCae C36:2))−0.5646 (Arg/PCae C34:2)−0.4139 (Age)−8.4352 (ln(BMI)).

For option (f) employing metabolite concentration SM C16:1 and the ratios PCaa C36:2 to PCae C36:2 and Trp to PCae C34:2 the equation is:

ln(odds of endometriosis)=57.953+0.4641(SM C16:1)−7.511(ln((PCaa C36:2/PCae C36:2))−2.7249 ln((Trp/PCae C34:2))−0.3811(Age)−7.7731(ln(BMI))

Finally, for option (g) employing the metabolite concentration SMOH C22:2 and the ratios of PCaa C36:2 to PCae C36:2 and Trp to PCae C34:0 the equation is:

ln(odds of endometriosis)=68.2797+0.1711(SMOH C22:2)−6.8735(ln(PCaa C36:2/PCae C36:2))−4.0881(ln((Trp.div.PC.ae.C34.0))−0.3893(Age)−6.1419(ln(BMI)).

The above described equations include a correct for age and BMI. However, the skilled person is well aware how to adjust such equations to be used without a correction for age and BMI.

From the thus calculated value, the odds of a possible presence of endometriosis for this subject can be derived, i.e. the odds of a possible presence of endometriosis=exp (ln (odds of endometriosis)).

Then, the probability for this subject of having endometriosis is calculated as follows: The probability of having endometriosis=odds of endometriosis/(1+odds of endometriosis).

TABLE 3
Cut-off values for probability of endometriosis. When the probability
calculated from the given combination is equal or higher than
the cut-off probability for this combination, the test should
be considered positive for endometriosis.

| | | | | Probability |
|---|---|---|---|---|
| | Metabolite | Ratio 1 | Ratio 2 | cut-off |
| (a) | SMOH C16:1 | PCaa C36:2/ | — | ≧0.58 |
| | | PCae C34:2 | | |
| (b) | PCae C30:0 | PCaa C36:2/ | Trp/PCae C34:0 | ≧0.466 |
| | | PCae C36:2 | | |
| (c) | PCae C36:1 | PCaa C36:2/ | Trp/PCae C34:0 | ≧0.50 |
| | | PCae C36:2 | | |
| (d) | SM C16:1 | PCaa C36:2/ | Trp/PCae C34:0 | ≧0.40 |
| | | PCae C36:2 | | |
| (e) | SM C16:1 | PCaa C36:2/ | Arg/PCae C34:2 | ≧0.46 |
| | | PCae C36:2 | | |
| (f) | SM C16:1 | PCaa C36:2/ | Trp/PCae C34:2 | ≧0.40 |
| | | PCae C36:2 | | |
| (g) | SMOH C22:2 | PCaa C36:2/ | Trp/PCae C34:0 | ≧0.47 |
| | | PCae C36:2 | | |

A probability of a higher value than the probability cut-off given in Table 3 is indicative of endometriosis. For example, a probability of 0.8 determined for option (a) is indicative for endometriosis as it is higher than the cut-off probability of 0.58 shown in Table 3.

In accordance with the present invention, an indication of endometriosis is given when (i) the concentration of the single metabolite measured in the sample is increased as compared to the value obtained from healthy subjects and (ii) Ratio 1 is decreased as compared to the value obtained from healthy subjects and, where a second ratio is determined, (iii) Ratio 2 is decreased as compared to the value obtained from healthy subjects.

In order to diagnose endometriosis in a caucasian woman, the skilled person may for example refer to the values as represented in tables 1 and 2 above or may calculate the probability based on the above given equations. Based on the data represented in tables 1 and 2, an indication of endometriosis can be given when (i) the concentration of the single metabolite measured is increased as compared to the cut-off value provided in table 2 and (ii) Ratio 1 is decreased as compared to the cut-off value provided in table 2 and, where a second ratio is determined, (iii) Ratio 2 is decreased as compared to the cut-off value provided in table 2. Similarly, where the above described equations are employed, an indication of endometriosis can be given when the probability of having endometriosis is above the probability cut-off shown in Table 3.

In accordance with the present invention, a non-invasive method for diagnosing ovarian endometriosis is provided, based on a measurement of a combination of metabolites in samples of patients. The present inventors analyzed the plasma metabolomes of endometriosis patients and compared them with the plasma metabolomes of healthy controls. The panel consisted of 148 metabolites including glycerophospholipids, sphingolipids and acylcarnitines. Eight lipids were identified as novel disease-associated biomarkers and 81 significantly different metabolite ratios were identified. The highest sensitivity and specificity was achieved by the measurement of the concentration of the hydroxysphingomyelin SMOH C16:1 and the ratio between phosphatidylcholine PCaa C36:2 to ether-phospholipid PCae C34:2. Also the combinations b) to g) were selected out of other potential combinations on the basis of a particularly high sensitivity (0.95) and specificity (0.94).

To the inventors best knowledge, the sensitivity and specificity parameters reported herein have not been reached with any other proposed markers (e.g. May et al., 2010; Mihalyi et al. 2010) nor with any of the methods presently employed in the art. Accordingly, the present invention provides an improved diagnostic test for endometriosis.

Besides the surprisingly high sensitivity and specificity, the present invention provides further advantageous properties. The sample, such as blood plasma can be easily obtained by a non-invasive method, thus providing an advantage over the currently established diagnostic approach that requires surgical visual inspection of the pelvic organs. Furthermore, the concentration of the selected metabolites can be determined using mass spectrometry, which provides superior selectivity and specificity.

In a preferred embodiment, the method further comprises normalising the obtained values.

In accordance with the present invention, the term “normalising the obtained values” relates to a correction of the measured value. This correction is usually carried out in order to adjust the values to patient-specific parameters and to control for bias introduced during the process of sample collection and analysis, which can, for example, arise due to variations based on different laboratories and/or different machines used. Importantly, normalisation enables a direct comparison of values obtained from individual patients.

Several strategies for the normalisation of metabolite concentrations are known in the art, including, without being limiting, normalising against the concentration of (an) internal reference, which is determined in the same sample, normalisation against sample size, normalisation against total metabolite amount or normalisation against an artificially introduced molecule of known amount. In addition, normalisation may also be carried out by adjusting the obtained values by patient-specific factors such as e.g. age, BMI, hormone status (e.g. menstrual cycle), nutritional factors (e.g. fasting) or time (circadian rhythm). Normalisation can for example be achieved by dividing the measured values of the metabolite to be investigated by the measured values of a reference molecule or by subtracting the measured values of the reference molecule from the measured value of the metabolite of interest.

In a more preferred embodiment, the normalisation is an adjustment for age and body mass index.

In order to adjust the measured metabolite concentrations for age, the woman's age in years is included in the above described equations.

In order to adjust the measured metabolite concentrations for body mass index (BMI), the woman's body mass index is calculated as the weight in kilograms (kg) divided by the square of the woman's height in meters (m²). The correction is carried out with including the BMI (kg/m²) in the above described equations, for examples as ln(BMI) as indicated above.

Where an adjustment for both age and BMI is carried out, both values are included in the above described equations.

As is shown in the appended examples, a higher age as well as a BMI higher than average have a protective effect. For example, as shown in Example 1, all participants classified into the underweight category (BMI<18.5 kg/m²; WHO criteria) were endometriosis patients and all of those classified into the obese category (BMI>30 kg/m²; Jekel, Katz and Elmore. 2001. Epidemiology, Biostatistics and Preventive Medicine, second edition. W.B. Saunders Company. p. 233), were healthy controls. By taking into account the factors age and body mass index (BMI), a surprisingly high sensitivity of 90.0% (85.0-92.5) and a specificity of at least 84.3% (84.3-92.2) could be reached, as shown in the appended examples.

In another preferred embodiment of the method of the invention, the concentrations are determined by mass spectrometry.

Mass spectrometry and its use for determining the concentration of metabolites in a sample is well known in the art and has been described for example in Griffiths W J, Koal T, Wang Y, Kohl M, Enot D P, Deigner H P (2010) Targeted metabolomics for biomarker discovery. Angew Chem Int Ed Engl 49: 5426-5445, Koal T, Deigner H P (2010) Challenges in mass spectrometry based targeted metabolomics. Curr Mol Med 10: 216-226.

Mass spectrometry includes, for example, tandem mass spectrometry, matrix assisted laser desorption ionization (MALDI) time-of-flight (TOF) mass spectrometry, MALDI-TOF-TOF mass spectrometry, MALDI Quadrupole-time-of-flight (Q-TOF) mass spectrometry, electrospray ionization (ESI)-TOF mass spectrometry, ESI-Q-TOF, ESI-TOF-TOF, ESI-ion trap mass spectrometry, ESI Triple quadrupole mass spectrometry, ESI Fourier Transform mass spectrometry (FTMS), MALDI-FTMS, MALDI-Ion Trap-TOF, and ESI-Ion Trap TOF.

At its most basic level, mass spectrometry involves ionizing a molecule and then measuring the mass of the resulting ions. Since molecules ionize in a way that is well known, the molecular weight of the molecule can be accurately determined from the mass of the ions. In addition, by a comparison of data obtained from internal standards, a quantification of molecules of interest is possible, as detailed herein below.

In a more preferred embodiment of the method of the invention, the mass spectrometry is selected from liquid chromatography mass spectrometry (LC-MS or HPLC-MS) and tandem mass spectrometry (MS-MS).

Liquid chromatography mass spectrometry combines the physical separation capabilities of liquid chromatography (LC) or high-performance liquid chromatography (HPLC), with the mass analysis capabilities of mass spectrometry (MS) (Kushnir M M, Rockwood A L, Roberts W L, Yue B, Bergquist J, Meikle A W (2011) Liquid chromatography tandem mass spectrometry for analysis of steroids in clinical laboratories. Clin Biochem 44: 77-88; Murray K K (2010) Glossary of terms for separations coupled to mass spectrometry. J Chromatogr A 1217: 3922-3928). HPLC provides the advantage over LC that has a shorter analysis time and better resolution of analytes. This consequently increases selectivity, precision and accuracy of MS.

Tandem mass spectrometry involves first obtaining a mass spectrum of the ion of interest, then fragmenting that ion and obtaining a mass spectrum of the fragments. Tandem mass spectrometry thus provides both molecular weight information and a fragmentation pattern that can be used in combination along with the molecular weight information to identify the exact sequence of a peptide or protein (see e.g. Hunt et al. (1986) PNAS USA 83:6233-6237; Shevchenko et al. (1996) PNAS USA 93:14440-14445; Figeys et al. (1996) Anal. Chem. 68:1822-1828 and Wilm et al. (1996) Nature 379:466-469).

In another preferred embodiment of the method of the invention, the concentrations are determined by reference to internal metabolite standards.

The abundance of any molecular ion species typically carries information on concentration, but ion abundance is confounded by a number of features, including instrument response factors, ionisation efficiency of the molecule, stability of the molecular ion species and the presence of other molecules that could cause ion suppression of the analyte of interest. Thus, internal standards have been developed that can be used to generate appropriate calibration curves to convert abundance of ions into a quantitative measure of metabolite concentration. Non-limiting examples of internal standards include metabolite standards labelled with stable isotope-labelled versions of the metabolite to be quantified, which have a similar extraction recovery, ionization response and a similar chromatographic retention time; compound analogues of the metabolite to be quantified which are similar to the compound to be quantified but slightly different by parent mass; or chlorinated versions of the metabolite to be quantified, which commonly have a similar chromatographic retention time.

The internal standard is typically added at a known concentration into every sample, including the standards, at the beginning of the sample preparation, typically before the plasma crash or solid phase extraction. It will be appreciated by the skilled person that the amount of the internal standard needs to be higher than the limit of quantitation but low enough to avoid a suppression of the ionization of the analyte. Based on the known concentration of the internal standard present in the sample, the measured values for the metabolite of interest can be quantified by interpolating the response ratio between the metabolite and the internal standard to a standard curve. These methods for determining metabolite concentrations by reference to internal metabolite standards are well known in the art and have been described, e.g. in Ciccimaro E, Blair I A (2010) Stable-isotope dilution LC-MS for quantitative biomarker analysis. Bioanalysis 2: 311-341; Koletzko B, Demmelmair H, Hartl W, Kindermann A, Koletzko S, Sauerwald T, Szitanyi P (1998) The use of stable isotope techniques for nutritional and metabolic research in paediatrics. Early Hum Dev 53 Suppl: S77-97; Postle A D, Hunt A N (2009) Dynamic lipidomics with stable isotope labelling. Journal of chromatography B, Analytical technologies in the biomedical and life sciences 877: 2716-2721).

In a more preferred embodiment, the internal metabolite standards are stable isotope-labelled standards.

Metabolite standards labelled with (a) stable isotope(s) are stable isotope-labelled versions of the metabolite to be quantified and are well known in the art. Typically, the isotopes employed are stable isotopes of carbon, nitrogen or hydrogen, such as e.g. ¹²C and ¹³C, ¹⁴N and ¹⁵N and ²H (Deuterium). Such stable isotope-labelled metabolite standards have been described e.g. in Lee et al. Clinical Biochemistry 43 (2010): 1269-1277.

In a preferred embodiment of the method of the invention, the sample is selected from blood, serum, plasma, saliva, urine, cerebrospinal fluid, condensates from respiratory air, tears, mucosal tissue, mucus, vaginal tissue, endometrium, including e.g. eutopic endometrium, skin, hair or hair follicle. In a more preferred embodiment of the method of the invention, the sample is selected from blood-serum or plasma.

In another preferred embodiment of the method of the invention, the subject is a human subject, preferably of caucasian race.

The present invention further relates to a kit comprising or consisting of
(a) stable isotope-labelled SMOH C16:1 or SMOH of a different mass and/or different a side chain desaturation level or a different desaturation position or a chemically similar compound not naturally occurring in the human sample; stable isotope-labelled PCaa C36:2 or PCaa of a different mass and/or a different side chain desaturation level or a different desaturation position or a chemically similar compound not naturally occurring in the human sample; and stable isotope-labelled PCae C34:2 or PCae of a different mass and/or a different side chain desaturation level or a different desaturation position or a chemically similar compound not naturally occurring in the human sample;
(b) stable isotope-labelled PCae C30.0 or PCae of a different mass and/or a different side chain desaturation level or a different desaturation position or a chemically similar compound not naturally occurring in the human sample; stable isotope-labelled PCaa C36.2 or PCaa of a different mass and/or a different side chain desaturation level or a different desaturation position or a chemically similar compound not naturally occurring in the human sample; stable isotope-labelled PCae C36.2 or PCae of a different mass and/or a different side chain desaturation level or a different desaturation position or a chemically similar compound not naturally occurring in the human sample; stable isotope-labelled Trp or a chemically similar compound not naturally occurring in the human sample; and stable isotope-labelled PCae C34.0 or PCae of a different mass and/or a different side chain desaturation level or a different desaturation position or a chemically similar compound not naturally occurring in the human sample;
(c) stable isotope-labelled PCae C36.1 or PCae of a different mass and/or a different side chain desaturation level or a different desaturation position or a chemically similar compound not naturally occurring in the human sample; stable isotope-labelled PCaa C36.2 or PCaa of a different mass and/or a different side chain desaturation level or a different desaturation position or a chemically similar compound not naturally occurring in the human sample; stable isotope-labelled PCae C36.2 or PCae of a different mass and/or a different side chain desaturation level or a different desaturation position or a chemically similar compound not naturally occurring in the human sample; stable isotope-labelled Trp or a chemically similar compound not naturally occurring in the human sample; and stable isotope-labelled PCae C34.0 or PCae of a different mass and/or a different side chain desaturation level or a different desaturation position or a chemically similar compound not naturally occurring in the human sample;
(d) stable isotope-labelled SM C16.1 or SM of a different mass and/or different a side chain desaturation level or a different desaturation position or a chemically similar compound not naturally occurring in the human sample; stable isotope-labelled PCaa C36.2 or PCaa of a different mass and/or a different side chain desaturation level or a different desaturation position or a chemically similar compound not naturally occurring in the human sample; stable isotope-labelled PCae C36.2 or PCae of a different mass and/or a different side chain desaturation level or a different desaturation position or a chemically similar compound not naturally occurring in the human sample; stable isotope-labelled Trp or a chemically similar compound not naturally occurring in the human sample; and stable isotope-labelled PCae C34.0 or PCae of a different mass and/or a different side chain desaturation level or a different desaturation position or a chemically similar compound not naturally occurring in the human sample;
(e) stable isotope-labelled SM C16.1 or SM of a different mass and/or different a side chain desaturation level or a different desaturation position or a chemically similar compound not naturally occurring in the human sample; stable isotope-labelled PCaa C36.2 or PCaa of a different mass and/or a different side chain desaturation level or a different desaturation position or a chemically similar compound not naturally occurring in the human sample; stable isotope-labelled PCae C36.2 or PCae of a different mass and/or a different side chain desaturation level or a different desaturation position or a chemically similar compound not naturally occurring in the human sample; stable isotope-labelled Arg or a chemically similar compound not naturally occurring in the human sample; and stable isotope-labelled PCae C34.2 or PCae of a different mass and/or a different side chain desaturation level or a different desaturation position or a chemically similar compound not naturally occurring in the human sample;
(f) stable isotope-labelled SM C16:1 or SM of a different mass and/or different a side chain desaturation level or a different desaturation position or a chemically similar compound not naturally occurring in the human sample; stable isotope-labelled PCaa C36:2 or PCaa of a different mass and/or a different side chain desaturation level or a different desaturation position or a chemically similar compound not naturally occurring in the human sample; stable isotope-labelled PCae C36:2 or PCae of a different mass and/or a different side chain desaturation level or a different desaturation position or a chemically similar compound not naturally occurring in the human sample; stable isotope-labelled Trp or a chemically similar compound not naturally occurring in the human sample; and stable isotope-labelled PCae C34:2 or PCae of a different mass and/or a different side chain desaturation level or a different desaturation position or a chemically similar compound not naturally occurring in the human sample; and/or
(g) stable isotope-labelled SMOH C22:2 or SMOH of a different mass and/or different a side chain desaturation level or a different desaturation position or a chemically similar compound not naturally occurring in the human sample; stable isotope-labelled PCaa C36:2 or PCaa of a different mass and/or a different side chain desaturation level or a different desaturation position or a chemically similar compound not naturally occurring in the human sample; stable isotope-labelled PCae C36:2 or PCae of a different mass and/or a different side chain desaturation level or a different desaturation position or a chemically similar compound not naturally occurring in the human sample; stable isotope-labelled Trp or a chemically similar compound not naturally occurring in the human sample; and stable isotope-labelled PCae C34:0 or PCae of a different mass and/or a different side chain desaturation level or a different desaturation position or a chemically similar compound not naturally occurring in the human sample.

SMOH, PCaa or PCae of a different mass and/or a side chain desaturation level or desaturation position include for example: PCaa C32:0, PCaa C32:1, PCaa C32:2, PCaa C32:3, PCaa C34:1, PCaa C34:3, PCaa C34:4, PCaa C36:3, PCaaC38:2 etc.

A chemically similar compounds not naturally occurring in the human sample include compounds that have for example a similar chemical formula, similar polarity or hydrophobicity, equal ionisation requirements, similar (but not identical) mass of precursor ion, and unique mass fragmentation spectrum. Non-limiting examples of chemically similar compounds not naturally occurring in the human sample include for example carbamyloxyphosphatidylcholine, diacetylene modified phospholipids, 1,2-Dimyristoyl-sn-glycero-3-phosphorylcholine, or lipids with non-natural chain length, all of which are well known in the art and have been described, e.g. in Curatolo W, Bali A, Gupta C M (1985) Phase behavior of carbamyloxyphosphatidylcholine, a sphingolipid analogue. J Pharm Sci 74: 1255-1258, Dagan A, Wang C, Fibach E, Gatt S (2003) Synthetic, non-natural sphingolipid analogs inhibit the biosynthesis of cellular sphingolipids, elevate ceramide and induce apoptotic cell death. Biochim Biophys Acta 1633: 161-169, Navab M, Hama S, Hough G, Fogelman A M (2003) Oral synthetic phospholipid (DMPC) raises high-density lipoprotein cholesterol levels, improves high-density lipoprotein function, and markedly reduces atherosclerosis in apolipoprotein E-null mice. Circulation 108: 1735-1739, Qin J D, Weiss L, Slavin S, Gatt S, Dagan A (2010) Synthetic, non-natural analogs of ceramide elevate cellular ceramide, inducing apoptotic death to prostate cancer cells and eradicating tumors in mice. Cancer Invest 28: 535-543, Zumbuehl A (2009) Nonnatural Pholpholipids: Probing Nature's Modular Platform. Chimia 63: 63-65.

Preferably, the kit comprises or consists of
(a) stable isotope-labelled SMOH C16:1; stable isotope-labelled PCaa C36:2 and stable isotope-labelled PCae C34:2;
(b) stable isotope-labelled PCae C30.0; stable isotope-labelled PCaa C36.2; stable isotope-labelled PCae C36.2; stable isotope-labelled Trp and stable isotope-labelled PCae C34.0;
(c) stable isotope-labelled PCae C36.1; stable isotope-labelled PCaa C36.2; stable isotope-labelled PCae C36.2; stable isotope-labelled Trp and stable isotope-labelled PCae C34.0;
(d) stable isotope-labelled SM C16.1; stable isotope-labelled PCaa C36.2; stable isotope-labelled PCae C36.2; stable isotope-labelled Trp and stable isotope-labelled PCae C34.0;
(e) stable isotope-labelled SM C16.1; stable isotope-labelled PCaa C36.2; stable isotope-labelled PCae C36.2; stable isotope-labelled Arg and stable isotope-labelled PCae C34.2;
(f) stable isotope-labelled SM C16:1; stable isotope-labelled PCaa C36:2; stable isotope-labelled PCae C36:2; stable isotope-labelled Trp and stable isotope-labelled PCae C34:2; and/or
(g) stable isotope-labelled SMOH C22:2; stable isotope-labelled PCaa C36:2; stable isotope-labelled PCae C36:2; stable isotope-labelled Trp and stable isotope-labelled PCae C34:0.

Most preferably, the kit comprises or consists of (i) stable isotope-labelled SMOH C16:1, (ii) stable isotope-labelled PCaa C36:2, and (iii) stable isotope-labelled PCae C34:2. Even more preferably, the kit consists of (i) stable isotope-labelled SMOH C16:1, (ii) stable isotope-labelled PCaa C36:2, and (iii) stable isotope-labelled PCae C34:2.

Stable isotope-labelled metabolite standards have been described herein above. The kit of the present invention comprises three such stable isotope-labelled metabolite standards, suitable for the quantification of metabolite concentrations of SMOH C16:1, PCaa C36:2 and PCae C34:2 in a sample, such as for example a sample obtained from a woman suspected of having endometriosis.

The preparation, general use and data interpretation of stable isotope-labelled lipids are well known in the art and stable-isotopes can be identified by their increase in mass and unique isotope distribution (Postle A D (2012) Lipidomics. Current opinion in clinical nutrition and metabolic care 15: 127-133, Postle A D, Hunt A N (2009) Dynamic lipidomics with stable isotope labelling. Journal of chromatography B, Analytical technologies in the biomedical and life sciences 877: 2716-2721).

Stable isotope-labelled SMOH C16:1 may for example involve ¹³C- and/or ¹⁵N- and/or ¹⁴N-serine residue(s) and/or ¹²C- and/or ¹³C in aliphatic chains giving rise to an unequivocal shift in mass pattern depending on the actual number of stable isotopes incorporated into the molecule.

Stable isotope-labelled PCaa C36:2 and stable isotope-labelled PCae C34:2 may employ ¹³C- and/or ¹⁵N- and/or ¹⁴N-choline residue(s) and/or ¹²C- and/or ¹³C in aliphatic chains giving rise to an unequivocal shift in mass pattern depending on the actual number of stable isotopes incorporated into the molecule.

The components of the kit may be packaged in one or more containers such as one or more vials. In addition to the metabolite standards, the kit preferably further comprises preservatives or buffers for storage. In addition, the kit may contain instructions for use.

In a preferred embodiment of the kit of the invention, the isotope is selected from the group consisting of ¹²C, ¹³C, ¹N, ¹⁵N and ²H.

The present invention further relates to the use of the kit of the invention in a method of diagnosing endometriosis in accordance with the present invention.

Unless otherwise defined, all technical and scientific terms used herein have the same meaning as commonly understood by one of ordinary skill in the art to which this invention pertains. In case of conflict, the present specification, including definitions, will control.

The figures show:

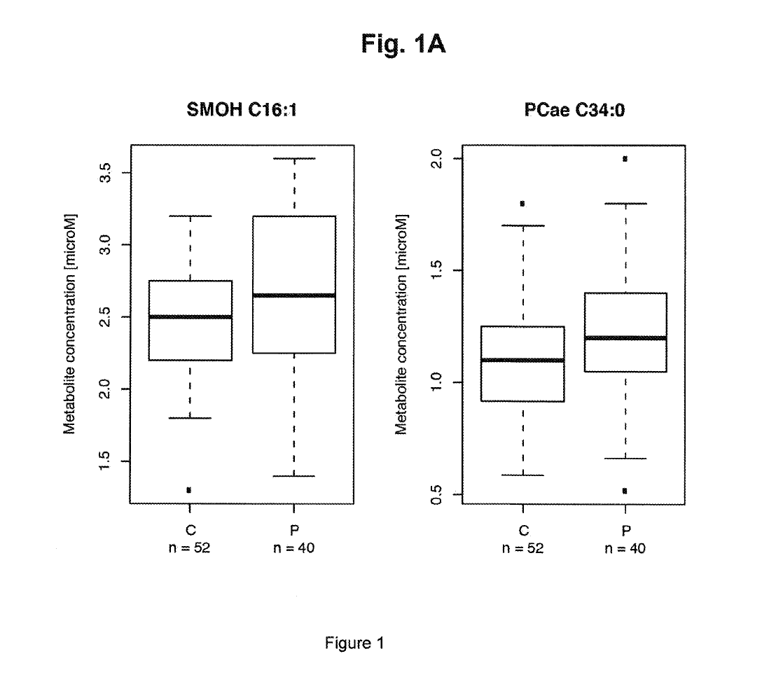

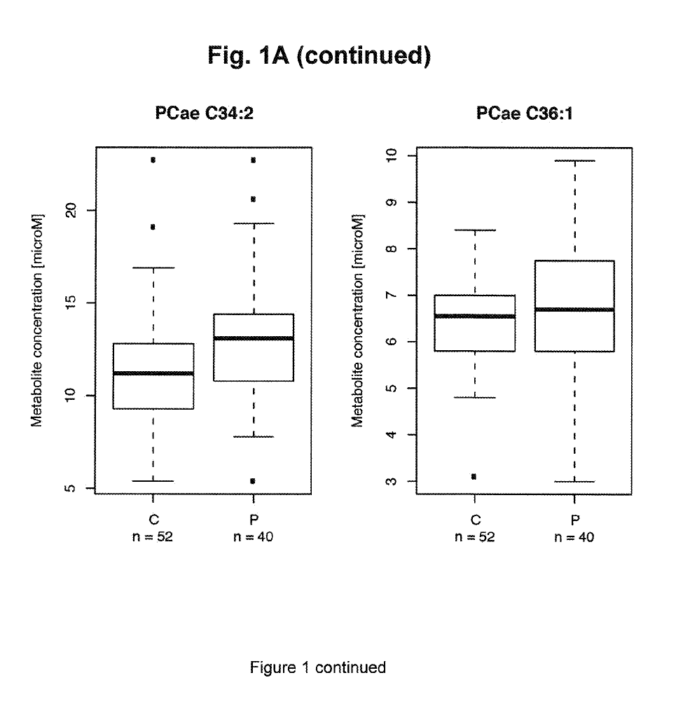

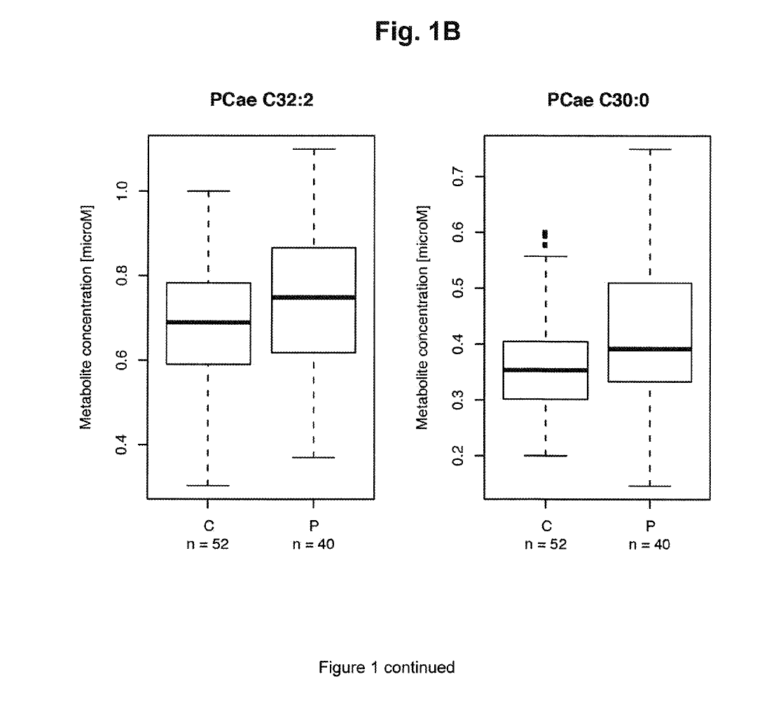

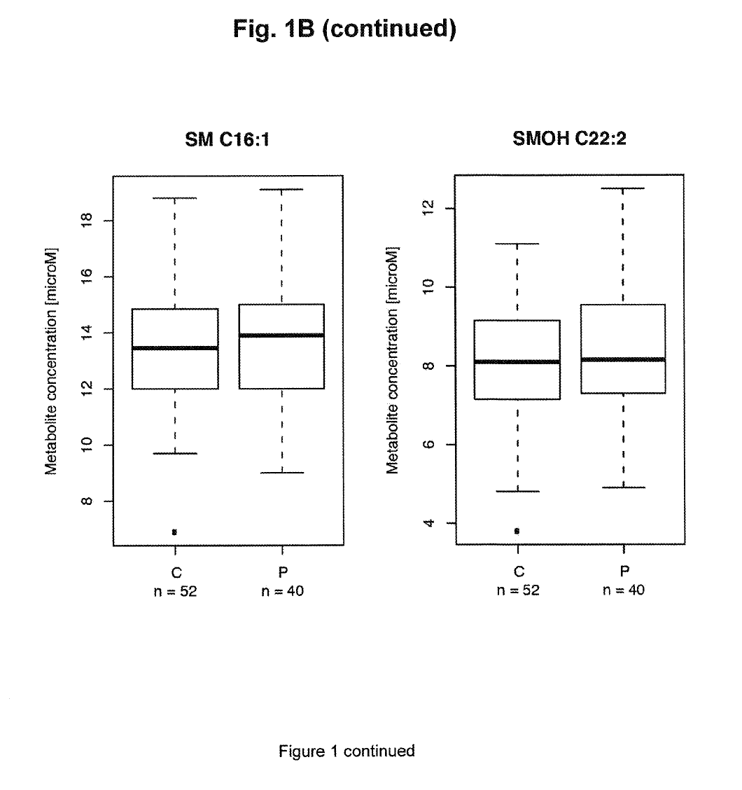

FIG. 1. Box plot for 8 metabolites.

Panels A and B: box plots of quartile distributions of eight metabolites for patients (P) and healthy controls (C).

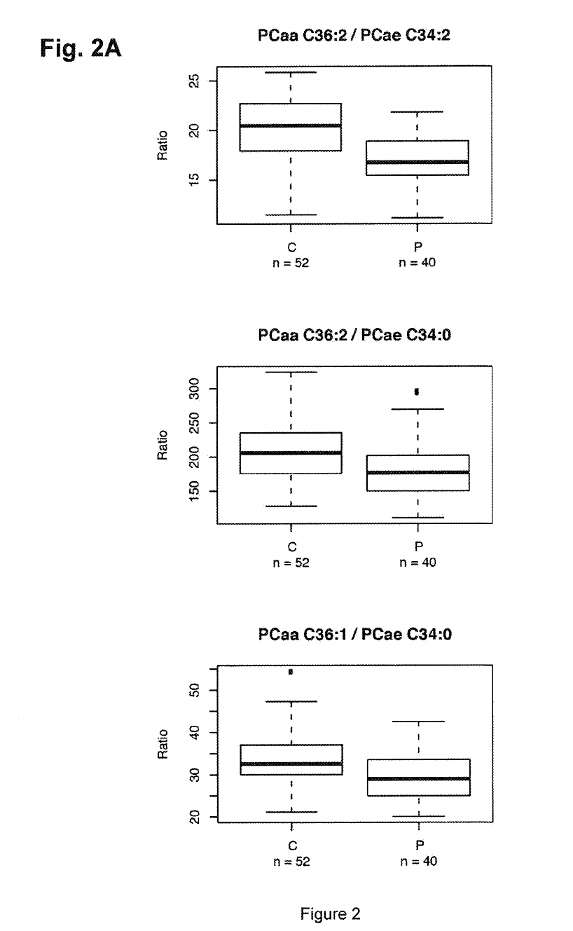

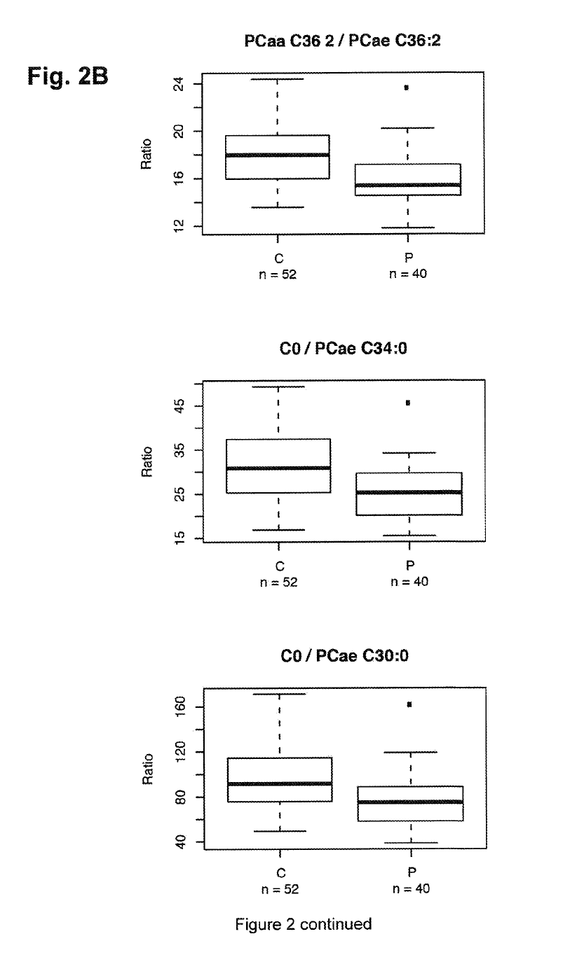

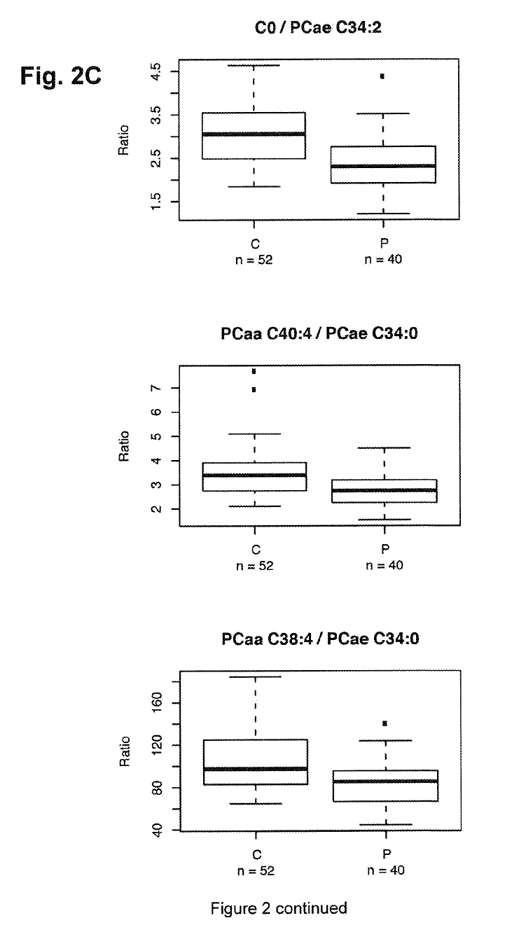

FIG. 2. Box plot for 9 selected metabolite ratios.

Panels A, B, and C: box plots of quartile distributions of nine selected metabolite ratios for patients (P) and healthy controls (C).

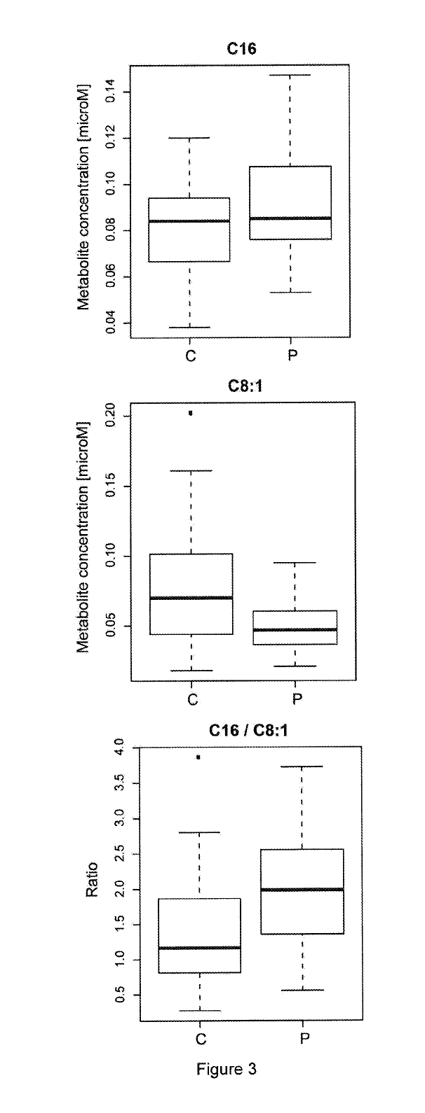

FIG. 3. Box plot for acylcarnitines C16, C8:1 and their ratio.

Box plots of quartile distributions for long-chain acylcarnitine C16, medium-chain acylcarnitine C8:1 and their ratio for patients (P) and healthy controls (C).

FIG. 4. ROC curve.

ROC curve shows improving effects of successive addition of separate variables to the model for differentiation between endometriosis patients and healthy controls.

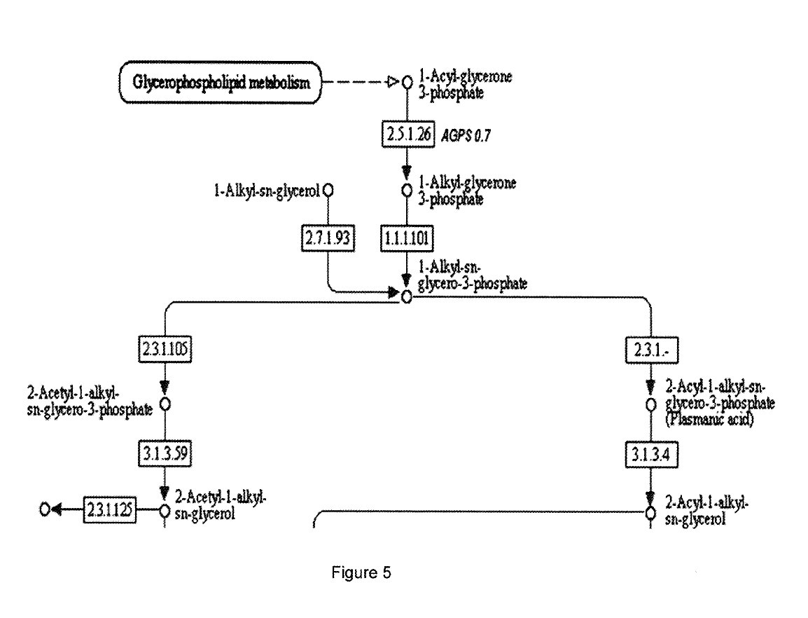

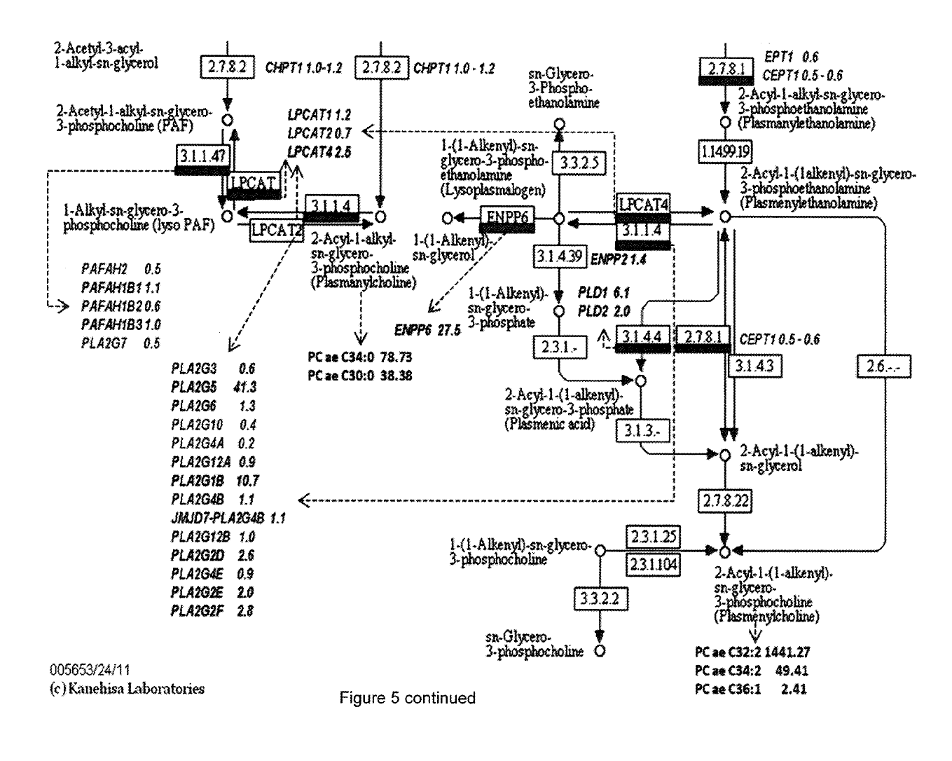

FIG. 5. Comparison of metabolic pathways and mRNA levels in endometriosis.

KEGG diagram (Kyoto Encyclopedia of Genes and Genomes, 2011) of ether lipid metabolism supplemented by data on ether-phospholipids increased in endometriosis (present results) as well as changed mRNA levels of the corresponding genes (the later were derived from data of Borghese et al. (2008)). Elevated ether-phospholipids are underlined (font: regular). Ether lipid species and their adjusted OR (present data, underlined font: regular) as well as enzyme names and fold changes in mRNA levels (Borghese et al., written in italics) are added to the right side or connected with blue arrows to the appropriate metabolite or enzyme box. Up-regulation is colored in black, down-regulation in gray.

The examples illustrate the invention:

### EXAMPLE 1

### Material and Methods

### Study Design and Sample Source

Patient enrolment took place from March 2008 to October 2009 at the Department of Obstetrics and Gynecology, University Clinical Centre Ljubljana, Slovenia. In a case-control study 111 probands were recruited from which a group of patients were selected with ovarian endometriosis that underwent laparoscopic surgery (n=40) and a control group of healthy women that underwent sterilization (n=52) and were surgically verified not to have endometriosis. On the day of surgery (prior to anesthesia), morning blood samples were collected from fasting participants and the participants were interviewed in order to obtain data on their ethnical origin, life style, gynecological and clinical conditions.

For blood collection and sample processing standard operating procedures were developed and implemented. Blood samples of 4 ml were obtained by venipuncture from median cubital vein using BD Vacutainer® tubes with K2 EDTA anticoagulant (Becton, Dickinson and Company, Franklin Lakes, US, #368861). Samples were turned upside-down for 8 to 10 times to allow for sufficient mixing with anticoagulant and put immediately at +4° C. The time of the sample collection was logged. Samples were collected within 1 hour and centrifuged at 1191 g for 10 minutes at +4° C. Plasma was aspirated and aliquots of 80 to 100 μl were stored at −80° C. in 1.8 ml cryotubes (Nalge Nunc International, Roskilde, Denmark, #375418). The time from sample collection to freezing varied between 1 to 2 hours. Aliquots used for measurements were transported on dry ice in a single batch to the measurement site.

Routine clinical biochemical parameters were measured at the Clinical Institute of Clinical Chemistry and Biochemistry of the University Clinical Centre Ljubljana, Slovenia. The study was approved by the National Medical Ethics Committee of the Republic of Slovenia, and all the participants signed a written informed consent before being enrolled in the study.

The diagnosis of endometriosis was confirmed histologically. Fourteen patients (35%) had only ovarian, 20 patients (50%) also peritoneal, and 6 patients (15%) had peritoneal and deep infiltrating endometriosis in addition to the ovarian. The majority of the patients had stage III or IV endometriosis. Staging of endometriosis was done according to the Revised American Society for Reproductive Medicine classification (Revised American Society for Reproductive Medicine, 1996). 19 probands out of 111 were excluded due to the following reasons: absence of ovarian endometriosis (11 patients), pregnancy (1 control), menopause (1 patient), surgery did not take place (2 controls) and errors in the sampling procedure (2 patients and 2 controls). The majority of the participants were not on reproductive tract related—hormonal therapy or oral contraception in the last 3 months before the therapy (62% of controls and 75% of patients) and none of them were on hormonal therapy or on oral contraception in the last week before surgery. The two study groups were well balanced in terms of comorbidities (presence of Uterus myomatosus or Myoma uteri), medication type as well as in proportion of participants that took/did not take any medication in the last week before surgery. The ethnical origin of the two study groups was similar. The majority of the study participants were of Slovene origin (both parents of the participants were Slovene). The two groups differed in their age structure. The age of the patients ranged between 22 to 44 years (mean age 33.3±6.06) and of the healthy controls between 32 to 45 years (mean age 40.6±3.1) (Table 4).

An inverse association between adult body mass index (BMI) and risk of endometriosis has been reported (reviewed in (Vitonis et al. 2010)). We observed the same tendency among our study participants; all participants classified into the underweight category were endometriosis patients and all of those classified into the obese category, were healthy controls. The BMI of the patients ranged between 17.01 to 28.58 (mean 20.90±2.72) and of the healthy controls between 18.83 to 33.90 (mean 25.68±4.05) (Table 4).

For estimating the phases of the menstrual cycle we used a proxy, calculated from the date of the last menstruation and corrected for an average length of the cycle. We classified the menstrual cycle phases of participants into proliferative, late proliferative/early secretory and secretory phase (Table 4).

### Metabolite Measurements

The targeted metabolomics approach was based on ESI-MS/MS measurements with the AbsoluteIDQ™ p150 kit (BIOCRATES Life Sciences AG, Innsbruck, Austria). The kit allows simultaneous quantification of 41 acylcarnitines (Cx:y), 92 glycerophospholipids (lysophosphatidylcholines (lysoPC) and phosphatidylcholines (PC)), 15 sphingolipids (SMx:y), 14 amino acids, and 1 hexose in a one step analysis. The assay procedures as well as the metabolite nomenclature have been previously described in detail (Gieger et al. 2008; Illig et al., 2010; Römisch-Margl et al., 2011). Quality assurance measures were done as already described (Illig et al., 2010). Quantification of the metabolites in the sample is achieved by reference to internal standards. The method has been proven to conform with 21CFR (Code of Federal Regulations) Part 11 FDA guidelines, which implies proof of reproducibility within a given error range.

### Statistical Analysis

Measurements were performed on three 96-well plates. The sample positions on the plates were randomly assigned. For estimating intra-plate and plate to plate variability, several duplicates of reference samples for each plate were used. For calculating coefficients of variation among replicated measurements, the metaP-server (Kastenmüller et al., 2011) was used. 14 amino acids and hexose were excluded, as they were not the focus of interest and 42 metabolites, where most of the measurements were below the LOD or coefficients of variation were over 0.25, were also excluded. Besides 106 metabolites that passed the quality control, all ratios between 5565 metabolite pairs ((n²−n)/2 ratios for n metabolites) were also analyzed.

The strength of association between the metabolites and the disease was first assessed with the non-parametric Mann-Whitney U-test, grouping participants according to disease status (case, control). To take account of multiple testing, the Benjamini-Hochberg false discovery rate correction procedure was applied (Benjamini and Hochberg, 1995) for performing 106 tests at the significance level of 5%.

Age and BMI were determined as the most important potential confounders or effect modifiers. Both study groups were well balanced in terms of distribution between phases of the menstrual cycle categories. However, the phase of the menstrual cycle is usually taken into account in endometriosis studies. Therefore, the influence of this variable was checked as well. The normality of distributions was assessed with the Shapiro-Wilk test and most of the metabolite and ratio variables were natural logarithmic transformed for further analyses. For assessing the strength of association between the metabolite or metabolite ratios and the disease, the odds ratio (OR) measure was used (Edwards et al., 1963). Missing values were excluded from non-descriptive analyses.

Crude associations (“crude OR”) between single metabolite concentrations or metabolite ratios and the disease were first assessed with a logistic regression basic model. For assessing the effect of potential confounders, the basic model was then extended by including the variables age, body mass index and phase of the menstrual cycle (multivariable logistic regression modeling). For every metabolite, a number of different models was tested including combinations of the three selected confounder variables as well as interactions between them. In case of most metabolites, inclusion of the phase of the menstrual cycle did not show significant confounding effect and did not improve the fit of the model. On the basis of the Akaike Information Criterion (AIC), the model which included age and body mass index was therefore selected as the best one. The model, which included a particular metabolite or ratio, adjusted for the effects of age and BMI, was used for calculating the “adjusted OR”.

As the selected metabolites belong to 4 classes of lipids (phosphatidylcholines, ether-phospholipids, acylcarnitines and sphongomylins) in 4 biosynthetic pathways, the correlation between the variables within the same lipid class is high. Adjustment of the significance level to p<0.01 was therefore considered being sufficient and the null hypotheses of the OR being equal to one where the p-values were <0.01 was rejected.

As more biomarkers substantially increase the reliability of a diagnostic/prognostic test, a backward stepwise-regression selection procedure was used to assess an impact of selected multiple metabolite variables. The data were processed with Microsoft Excel 2003. For statistical analyses, R 2.11.1 (R Development Core Team, 2008), R-package Epicalc (Chongsuvivatwong, 2010) and SAS 9.1 (SAS Institute Inc: SAS/STAT Users Guide, Version 9.1. Cary N.C.: SAS Institute Inc; 2003) were used.

### Data Mining

Due to the neovascularisation around and within endometriosis lesions (Taylor et al., 2009), it was postulated that many changes within ectopic endometrium could be reflected in blood plasma of the patients. Additionally to analyzing plasma metabolites, the gene expression results of Borghese et al. (NCBI GEO database, accession number GSE12768) (Borghese et al., 2008) were used as help for interpretation of the data. The design of the latter study differed from the present study as the control tissue originated from the same patients. However, as the focus of their and the present study was advanced stage ovarian endometriosis, it was considered that this dataset is a suitable source for looking up expression of enzymes in potentially affected biochemical pathways.

### EXAMPLE 2

### Eight Metabolites were Elevated in Plasma of Patients Compared to Controls

The plasma metabolites of endometriosis patients and healthy controls were quantified in a targeted metabolomics approach. In the first step differences in concentrations of single metabolites between the two study groups were looked at. Crude as well as for age and BMI adjusted OR were calculated with corresponding 95% confidence intervals for 106 metabolites, which passed measurement quality control. After correction for age and BMI, eight metabolites showed significant differences (p<0.01) between patients and controls (FIG. 1). Among them were the hydroxysphingomyelins SMOH C16:1 and SMOH C22:2, the sphingomyelin SMC16:1 and five ether-phospholipids (acyl-alkyl-phosphatidylcolines): three unsaturated 2-acyl-1-(1-alkenyl)-sn-glycero-3-phosphocholines (plasmenycholines), PCae C32:2, PCae C34:2, and PCae C36:1 as well as two saturated 2-acyl-1-alkyl-sn-glycero-3-phosphocholines (plasmanylcholines), PCae C34:0 and PCae C30:0. OR higher than 1, designate, that all 8 metabolites were elevated in plasma of patients compared to controls (Table 5). None of the 8 confidence intervals included 1 and the lower limits of the 95% confidence intervals were reasonably higher than 1 for at least the first 6 metabolites, therefore it can be concluded that the observed differences are not due to chance.

After correction for multiple testing, univariate analysis showed significantly lower values in patients for 3 metabolites: medium-chain acylcarnitine C8:1, phospholipid PCaa C38:4 and free carnitine CO with respective p-values of 0.00032, 0.00085 and 0.00124. The result was confirmed by logistic regression (crude OR). In addition to these three metabolites, crude OR showed also that phospholipid PCaa C38:3 was significantly lower (OR<1) and long-chain acylcarnitine C18 and ether-phospholipid PCae C42:3 were significantly higher (OR>1) in patients. However, the differences were no longer significant after adjustment for age and BMI (data not shown). Either or both, age and BMI, showed strong confounding effects on associations of these metabolites with the disease.

### EXAMPLE 3

### Eighty-One Metabolite Ratios Showed Significant Differences Between Patients and Controls

Besides differences in single metabolite concentrations, ratios between pairs of metabolite concentrations were also analyzed. They served primarily as sources of additional information on affected biochemical pathways. Crude as well as for age and BMI adjusted OR were calculated with corresponding 95% confidence intervals for all 5565 metabolite pairs. After correction for age and BMI, 81 ratios showed significant differences between patients and controls (p<0.01).

Being aware of limitations due to the study sample size, only the 9 most significantly different ratios were taken into consideration for the further biomarker selection procedure (FIG. 2). Interestingly, different metabolites such as diacyl-phosphatidylcholines and free carnitine (CO) were observed in the numerator but only ether-phospholipids (acyl-alkyl-phosphatidylcholines) in the denominator (Table 5). None of the 9 confidence intervals included 1 and the upper limits of the 95% confidence intervals were reasonably lower than 1.

The ratio between long-chain acylcarnitine C16 and medium-chain acylcarnitine C8:1 does not belong to the 9 most significantly different ratios (FIG. 3). However, it appeared attractive as it was one of the rare elevated ratios in patient's. In order to see whether this represents a general tendency between long- and medium-chain acylcarnitines, also the other ratio pairs of long-chain acylcarnitines over medium-chain acylcarnitine C8:1 were examined (Table 7). They all turned out to be elevated in patients, however after correction for age and BMI the differences were no longer significant (p>0.01). Pearson correlation coefficients between these ratios and the number of leucocytes in blood, an indicator of inflammation were calculated. The correlation was positive for all 8 ratios and significant (p<0.01) for 5 out of 8.

### EXAMPLE 4

### Hydroxysphingomyelin C16:1 and the Ratio Between Phosphatidylcholine C36:2 to Ether-Phospholipid C34:2 Represent a Potential Biomarker Combination

In a next step, the impact of multiple metabolites as well as ratios was assessed and the best combination of potential biomarkers for endometriosis diagnostics was selected, using a backward stepwise-regression selection procedure. The model, containing hydroxysphingomyelin SMOH C16:1 and the ratio between phosphatidylcholine PCaa C36:2 to ether-phospholipid PCae C34:2, corrected for the effect of age and BMI, showed the best characteristics (Table 8). By assuming a 10% prevalence of endometriosis in women population, 90.0% sensitivity, 84.3% specificity, likelihood ratio positive (LR+) of 5.7, likelihood ratio negative (LR−) of 0.1 and a ratio of LR+ to LR− of 48.3 for the given combination of variables was found. The receiver operating characteristic (ROC) curve shows improving effects of adding separate variables to the model. The combination of all four variables results in a significantly better performing curve and allows a very good discrimination between patients and healthy controls (FIG. 4).

TABLE 4
Characteristics of the study participants

| | Controls n = 52 | | Patients n = 40 | |
|---|---|---|---|---|
| | Frequency | % | Frequency | % |
| Age Category | | | | |
| <26 years | 0 | 0.00 | 5 | 12.50 |
| 26-29.9 years | 0 | 0.00 | 9 | 22.50 |
| 30-35.9 years | 3 | 5.77 | 12 | 30.00 |
| 36-40.9 years | 22 | 42.31 | 9 | 22.50 |
| >41 years | 27 | 51.92 | 5 | 12.50 |
| missing | 0 | 0 | 0 | 0 |
| BMI category | | | | |
| underweight <18.5 | 0 | 0.00 | 8 | 20.00 |
| normal 18.6-24.9 | 23 | 44.23 | 29 | 72.50 |
| overweight 25-29.9 | 20 | 38.46 | 3 | 7.50 |
| obese >30 | 8 | 15.38 | 0 | 0.00 |
| missing | 1 | 1.92 | 0 | 0.00 |
| Menstrual phase | | | | |
| proliferative | 17 | 32.69 | 12 | 30.00 |
| late prol./early sec. | 11 | 21.15 | 8 | 20.00 |
| secretory | 21 | 40.38 | 20 | 50.00 |
| not determined* | 2 | 3.85 | 0 | 0.00 |
| missing | 1 | 1.92 | 0 | 0.00 |
| Ethnicity** | | | | |
| Slovene | 35 | 67.31 | 28 | 70.00 |
| Slovene-foreign | 1 | 1.92 | 7 | 17.50 |
| foreign | 13 | 25.00 | 5 | 12.50 |
| missing | 3 | 5.77 | 0 | 0.00 |
| Medication in the week | | | | |
| before*** | | | | |
| no | 33 | 63.46 | 26 | 65.00 |
| yes | 16 | 30.77 | 14 | 35.00 |
| missing | 3 | 5.77 | 0 | 0.00 |
| Concomitant diseases | | | | |
| Adenomyosis | 0 | 0.00 | 1 | 2.50 |
| Myoma uteri or Uterus | 3 | 5.77 | 5 | 12.50 |
| myomatosus | | | | |
| none | 48 | 92.31 | 34 | 85.00 |
| missing | 1 | 1.92 | 0 | 0.00 |

*For two study participants we could not determine the phase of the menstrual cycle as they were on oral contraception until the last week before the surgery.
**Origin of parents.
***Allowed medications had the half-lives up to 26 h, the only exception was levothyroxine taken by 2 patients and 1 control.

TABLE 5
OR for metabolites elevated in endometriosis.

| | | | | | 95% CI | |
|---|---|---|---|---|---|---|
| | | Crude | Adjusted | 95% CI lower | upper | p-value |
| | Metabolite | OR | OR | (adj. OR) | (adj. OR) | (adj. OR) |
| 1 | SMOH C16:1 | 2.38E+00 | 1.70E+01 | 2.90E+00 | 9.93E+01 | 1.69E−03 |
| 2 | PCae C34:0 | 4.48E+00 | 7.87E+01 | 4.69E+00 | 1.32E+03 | 2.41E−03 |
| 3 | PCae C32:2 | 1.55E+01 | 1.44E+03 | 1.06E+01 | 1.95E+05 | 3.68E−03 |
| 4 | PCae C30:0 | 5.63E+00 | 3.84E+01 | 3.25E+00 | 4.53E+02 | 3.76E−03 |
| 5 | PCae C34:2 | 6.96E+00 | 4.94E+01 | 3.39E+00 | 7.20E+02 | 4.32E−03 |
| 6 | PCae C36:1 | 1.22E+00 | 2.41E+00 | 1.28E+00 | 4.54E+00 | 6.65E−03 |
| 7 | SM C16:1 | 9.98E−01 | 1.73E+00 | 1.15E+00 | 2.59E+00 | 8.48E−03 |
| 8 | SMOH C22:2 | 1.10E+00 | 2.00E+00 | 1.18E+00 | 3.39E+00 | 9.77E−03 |

TABLE 6
OR for the 9 most significantly different ratios.

| | | | | 95% CI | 95% CI | |
|---|---|---|---|---|---|---|
| | | | | lower (adj. | upper (adj. | P-value |
| | Ratio | Crude OR | Adjusted OR | OR) | OR) | (adj. OR) |
| 1 | PCaa C36:2/PCae | 6.83E−01 | 6.12E−01 | 4.63E−01 | 8.08E−01 | 5.40E−04 |
| | C34:2 | | | | | |
| 2 | PCaa C36:2/PCae | 9.28E−04 | 7.13E−05 | 2.41E−07 | 2.11E−02 | 1.01E−03 |
| | C36:2 | | | | | |
| 3 | C0/PCae | 1.28E−02 | 7.85E−03 | 4.14E−04 | 1.49E−01 | 1.24E−03 |
| | C34:2 | | | | | |
| 4 | PCaa C36:2/PCae | 4.67E−02 | 3.80E−03 | 1.26E−04 | 1.14E−01 | 1.33E−03 |
| | C34:0 | | | | | |
| 5 | C0/PCae | 4.01E−02 | 9.14E−03 | 4.86E−04 | 1.72E−01 | 1.72E−03 |
| | C34:0 | | | | | |
| 6 | PCaa C40:4/PCae | 3.24E−02 | 6.25E−03 | 2.58E−04 | 1.51E−01 | 1.79E−03 |
| | C34:0 | | | | | |
| 7 | PCaa C36:1/PCae | 4.31E−02 | 3.99E−03 | 1.21E−04 | 1.32E−01 | 1.97E−03 |
| | C34:0 | | | | | |
| 8 | C0/PCae | 4.34E−02 | 2.04E−02 | 1.67E−03 | 2.49E−01 | 2.31E−03 |
| | C30:0 | | | | | |
| 9 | PCaa C38:4/PCae | 3.00E−02 | 4.07E−03 | 1.16E−04 | 1.42E−01 | 2.40E−03 |
| | C34:0 | | | | | |

TABLE 7
OR for the ratios between long-chain acylcarnitines and the
medium-chain acylcarnitine C8:1 elevated in endometriosis.

| | | | | 95% CI | 95% CI | | | |
|---|---|---|---|---|---|---|---|---|
| | | | | lower | upper | p-value | | Corr. test |
| | Ratios | Crude OR | Adjusted OR | (adj. OR) | (adj. OR) | (adj. OR) | r | p-values |
| 1 | C16/C8:1 | 6.93E+00 | 9.29E+00 | 1.83E+00 | 4.71E+01 | 7.12E−03 | 0.29 | 5.93E−03 |
| 2 | C14/C8:1 | 6.14E+00 | 7.99E+00 | 1.60E+00 | 3.99E+01 | 1.14E−02 | 0.40 | 9.00E−05 |
| 3 | C18/C8:1 | 6.80E+00 | 5.69E+00 | 1.47E+00 | 2.21E+01 | 1.20E−02 | 0.18 | 7.81E−02 |
| 4 | C14:1 OH/C8:1 | 6.80E+00 | 6.69E+00 | 1.41E+00 | 3.18E+01 | 1.69E−02 | 0.29 | 6.23E−03 |
| 5 | C16:2/C8:1 | 3.82E+00 | 4.78E+00 | 1.32E+00 | 1.74E+01 | 1.74E−02 | 0.39 | 1.10E−04 |
| 6 | C18:2/C8:1 | 4.78E+00 | 4.42E+00 | 1.20E+00 | 1.62E+01 | 2.51E−02 | 0.14 | 1.73E−01 |
| 7 | C18:1/C8:1 | 5.79E+00 | 4.66E+00 | 1.16E+00 | 1.87E+01 | 2.97E−02 | 0.23 | 3.27E−02 |
| 8 | C12/C8:1 | 4.47E+00 | 3.62E+00 | 1.02E+00 | 1.28E+01 | 4.62E−02 | 0.37 | 3.20E−04 |

The Pearson correlation coefficient between these ratios and the number of leucocytes in blood is designated as “r”.

TABLE 8
Final combination of the best potential biomarkers of endometriosis.

| | | | 95% CI | 95% CI | |
|---|---|---|---|---|---|
| | | Adjusted | lower (adj. | upper (adj. | p-value |
| | Crude OR | OR | OR) | OR) | (adj. OR) |
| SM OH | 2.38E+00 | 9.46E+00 | 1.41E+00 | 6.37E+01 | 2.09E−02 |
| C16:1 | | | | | |
| PC aa C36:2/ | 6.83E−01 | 6.48E−01 | 4.79E−01 | 8.77E−01 | 4.89E−03 |
| PC ae C34:2 | | | | | |
| Age | 7.29E−01 | 7.08E−01 | 5.83E−01 | 8.60E−01 | 4.97E−04 |
| BMI | 8.54E−05 | 1.03E−03 | 5.10E−06 | 2.08E−01 | 1.11E−02 |

### REFERENCES

Agwu D E, McPhail L C, Chabot M C, Daniel L W, Wykle R L, McCall C E. Choline-linked phosphoglycerides. A source of phosphatidic acid and diglycerides in stimulated neutrophils. J Biol Chem 1989; 264:1405-1413.
Benjamini Y, Hochberg Y. Controling false discovery rate: a practical and powerful approach to multiple testing. J R Statist Soc B 1995; 57:289-300.
Biancone L, Cantaluppi V, Del Sorbo L, Russo S, Tjoelker L W, Camussi G. Platelet-activating factor inactivation by local expression of platelet-activating factor acetyl-hydrolase modifies tumor vascularization and growth. Clin Cancer Res 2003; 9:4214-4220.
Billah M M, Eckel S, Mullmann T J, Pai J K, Siegel M I, Egan R W. Phosphatidylcholine hydrolysis by phospholipase D determines phosphatidate and diglyceride levels in chemotactic peptide-stimulated human neutrophils. Involvement of phosphatidate phosphohydrolase in signal transduction. J Biol Chem 1989; 264:17069-17077.
Bland M. Introduction to medical statistics. Third edition. 2000. Oxford University Press Inc. New York, USA. pp 322-323.
Bolton E, Wang Y, Thiessen P A, Bryant S H. PubChem: Integrated Platform of Small Molecules and Biological Activities. Chapter 12 I N Annual Reports in Computational Chemistry, Volume 4, 2008. American Chemical Society, Washington, D.C., USA.
Borghese B, Mondon F, Noel J C, Fayt I, Mignot T_{M}, Vaiman D, Chapron C. Gene expression profile for ectopic versus eutopic endometrium provides new insights into endometriosis oncogenic potential. Mol Endocrinol 2008; 22:2557-2562.
Brosens I, Puttemans P, Campo R, Gordts S, Brosens J (2003) Non-invasive methods of diagnosis of endometriosis. Curr. Opin. Obstet. Gynecol 15: 519-522.
Bussolino F, Camussi G. Platelet-activating factor produced by endothelial cells. A molecule with autocrine and paracrine properties. Eur J Biochem 1995; 229:327-337.
ChemIDPlus Advanced [Internet]. Bethesda (Md.): National Library of Medicine (U S), National Institutes of Health 2003 [last updated Feb. 9, 2011]. (http://chem.sis.nlm.nih.gov/chemidplus/chemidheavy.jsp). Accessed: Jan. 2, 2012.
Chongsuvivatwong V. epicalc: Epidemiological calculator. 2010. (http://CRAN.R-project.org/package=epicalc)
Cuvillier O. Sphingosine in apoptosis signaling. Biochim Biophys Acta 2002; 1585:153-162.
D'Hooghe T, Hummelshoj L. Multi-disciplinary centres/networks of excellence for endometriosis management and research: a proposal. Hum Reprod 2006; 21:2743-2748.
Dionyssopoulou E, Vassiliadis S, Evangeliou A, Koumantakis E E, Athanassakis I. Constitutive or induced elevated levels of L-carnitine correlate with the cytokine and cellular profile of endometriosis. J Reprod Immunol 2005; 65:159-170.
Daniel L W, Waite M, Wykle R L. A novel mechanism of diglyceride formation. 12-O-tetradecanoylphorbol-13-acetate stimulates the cyclic breakdown and resynthesis of phosphatidylcholine. J Biol Chem 1986; 261:9128-9132.
Edwards A W F. The Measure of Association in a 2×2 Table. J R Stat Soc Series A Stat Methodol. J R Stat Soc. Series A General. 1963; 126:109-114.
Gebel H M, Braun D P, Tambur A, Frame D, Rana N, Dmowski W P. Spontaneous apoptosis of endometrial tissue is impaired in women with endometriosis. Fertil Steril 1998; 69:1042-1047.
Gieger C, Geistlinger L, Altmaier E, Hrabé de Angelis M, Kronenberg F, Meitinger T, Mewes H W, Wichmann H E, Weinberger K M, Adamski J. et al. Genetics meets metabolomics: a genome-wide association study of metabolite profiles in human serum. PLoS Genet 2008; 4:e1000282.
Giudice L C, Kao L C. Endometriosis. Lancet 2004; 364:1789-1799. Hadfield R, Mardon H, Barlow D, Kennedy S. Delay in the diagnosis of endometriosis: a survey of women from the USA and the U K. Hum Reprod 1996; 11:878-880.
Hii C S, Kokke Y S, Pruimboom W, Murray A W. Phorbol esters stimulate a phospholipase D-catalysed reaction with both ester- and ether-linked phospholipids in HeLa cells. FEBS Letters 1989; 257:35-37.
Illig T, Gieger C, Zhai G, Römisch-Margl W, Wang-Sattler R, Prehn C, Altmaier E, Kastenmüller G, Kato B S, Mewes H W, et al. A genome-wide perspective of genetic variation in human metabolism. Nat Genet 2010; 42:137-141.
Jekel J F, Katz D L, Elmore J G. Epidemiology, biostatistics and preventive medicine. Second edition. 2001 W.B. Saunders Company, An imprint of Elsevier Science. Philadelphia, Pa., USA. pp 106-113.
Kastenmüller G, Römisch-Margl W, Wagele B, Altmaier E, Suhre K. metaP-server: a web-based metabolomics data analysis tool. J Biomed Biotechnol 2011; 2011 pii: 839862.
KEGG: Kyoto Encyclopedia of Genes and Genomes. (http://www.genome.jp/kegg/). (Accessed May 20, 2011)
Kharfi A, Akoum A. Soluble interleukin-1 receptor type II blocks monocyte chemotactic protein-1 secretion by U937 cells in response to peripheral blood serum of women with endometriosis. Fertil Steril 2002; 78:836-842.
Ko H M, Jung H H, Seo K H, Kang Y R, Kim H A, Park S J, Lee H K, Im S Y. Platelet-activating factor-induced N F-kappaB activation enhances VEGF expression through a decrease in p53 activity. FEBS Lett 2006; 580:3006-3012.
Li H, Fukuda S, Hasegawa Y, Purevsuren J, Kobayashi H, Mushimoto Y, Yamaguchi S. Heat stress deteriorates mitochondrial beta-oxidation of long-chain fatty acids in cultured fibroblasts with fatty acid beta-oxidation disorders. J Chromatogr B Analyt Technol Biomed Life Sci 2010; 878:1669-1672.
May K E, Conduit-Hulbert S A, Villar J, Kirtley S, Kennedy S H, Becker C M. Peripheral biomarkers of endometriosis: a systematic review. Hum Reprod Update 2010; 16:651-674.
Mandal A, Wang Y, Ernsberger P, Kester M. Interleukin-1-induced ether-linked diglycerides inhibit calcium-insensitive protein kinase C isotypes. Implications for growth senescence. J Biol Chem 1997; 272:20306-20311.
Mansour G, Abdelrazik H, Sharma R K, Radwan E, Falcone T, Agarwal A. L-carnitine supplementation reduces oocyte cytoskeleton damage and embryo apoptosis induced by incubation in peritoneal fluid from patients with endometriosis. Fertil Steril 2009; 91:2079-2086.
Mechsner S, Grum B, Gericke C, Loddenkemper C, Dudenhausen J W, Ebert A D. Possible roles of oxytocin receptor and vasopressin-1α receptor in the pathomechanism of dysperistalsis and dysmenorrhea in patients with adenomyosis uteri. Fertil Steril 2010; 94:2541-2546.
Mihalyi A, Gevaert O, Kyama C M, Simsal P, Pochet N, De Smet F, De Moor B, Meuleman C, Billen J, Blanckaert N, Vodolazkaia A, Fulop V, and D'Hooghe T_{M}, (2010) Non-invasive diagnosis of endometriosis based on a combined analysis of six plasma biomarkers. Hum. Reprod., 25: 654-664.
Mier-Cabrera J, Jimenez-Zamudio L, Garcia-Latorre E, Cruz-Orozco O, Hernandez-Guerrero C. Quantitative and qualitative peritoneal immune profiles, T-cell apoptosis and oxidative stress-associated characteristics in women with minimal and mild endometriosis. BJOG. 2011; 118:6-16.
Mol B W, Bayram N, Lijmer J G, Wiegerinck M A, Bongers M Y, et al. (1998) The performance of C A-125 measurement in the detection of endometriosis: a meta-analysis. Fertil. Steril 70: 1101-1108.
Murphy R, Nishijima M, Raetz C, Shimizu T, Spener F, van Meer G, Wakelam M, Dennis E Update of the LIPID MAPS comprehensive classification system for lipids. Journal of Lipid Research 2009; 50, S9-S14.
Nagan N, Zoeller R A. Plasmalogens: biosynthesis and functions. Prog Lipid Res 2001; 40:199-229.
Prescott S M, Zimmerman G A, Stafforini D M, McIntyre T_{M}. Platelet-activating factor and related lipid mediators. Annu Rev Biochem 2000; 69:419-445.
Quinn M. Endometriosis: the consequence of neurological dysfunction? Med Hypotheses 2004; 63:602-608.
Revised American Society for Reproductive Medicine classification of endometriosis: 1996. Fertil Steril 1997; 67:817-821.
R Development Core Team RDCT. A language and environment for statistical computing. 2008; (http://www.R-project.org)
Römisch-Margl W, Prehn C, Bogumil R, Röhring C, Suhre K and Adamski J. Procedure for tissue sample preparation and metabolite extraction for high-throughput targeted metabolomics. Metabolomics 2011 DOI: 10.1007/s11306-011-0293-40
Taylor R N, Yu J, Torres P B, Schickedanz A C, Park J K, Mueller M D, Sidell N. Mechanistic and Therapeutic Implications of Angiogenesis in Endometriosis. Reprod Sci 2009; 16:140-146.
Velasco I, Acién P, Campos A, Acién M I, Ruiz-Maciá E. Interleukin-6 and other soluble factors in peritoneal fluid and endometriomas and their relation to pain and aromatase expression. J Reprod Immunol 2010; 84:199-205.
Vitonis A F, Baer H J, Hankinson S E, Laufer M R, Missmer S A. A prospective study of body size during childhood and early adulthood and the incidence of endometriosis. Hum Reprod 2010; 25:1325-1334.
Vouk K, Smuc T, Guggenberger C, Ribi\check{c}-Pucelj M, Sinkovec J, Husen B, Thole H, Houba P, Thaete C, Adamski J et al. Novel estrogen-related genes and potential biomarkers of ovarian endometriosis identified by differential expression analysis. J Steroid Biochem Mol Biol 2011; 125:231-42.
Wishart D S, Tzur D, Knox C, Eisner R, Guo A C, Young N, Cheng D, Jewell K, Arndt D, Sawhney S et al. HMDB: the Human Metabolome Database. Nucleic Acids Res 2007; 35(Database issue):D521-6.
Wishart D S, Knox C, Guo A C, Eisner R, Young N, Gautam B, Hau D D, Psychogios N, Dong E, Bouatra S et al. HMDB: a knowledgebase for the human metabolome. Nucleic Acids Res 2009; 37(Database issue):D603-10.

1. A method of diagnosing endometriosis or for producing diagnostically informative concentration values of metabolites for endometriosis, the method comprising
(i) determining in a sample obtained from a subject:
(a) the concentration of SMOH C16:1 and the ratio of the concentration of PCaa C36:2 to the concentration of PCae C34:2;
(b) the concentration of PCae C30.0, the ratio of the concentration of PCaa C36.2 to the concentration of PCae C36.2 and the ratio of the concentration of Trp to the concentration of PCae C34.0;
(c) the concentration of PCae C36.1, the ratio of the concentration of PCaa C36.2 to the concentration of PCae C36.2 and the ratio of the concentration of Trp to the concentration of PCae C34.0;
(d) the concentration of SM C16.1, the ratio of the concentration of PCaa C36.2 to the concentration of PCae C36.2 and the ratio of the concentration of Trp to the concentration of PCae C34.0;
(e) the concentration of SM C16.1, the ratio of the concentration of PCaa C36.2 to the concentration of PCae C36.2 and the ratio of the concentration of Arg to the concentration of PCae C34.2;
(f) the concentration of SM C16:1, the ratio of the concentration of PCaa C36:2 to the concentration of PCae C36:2 and the ratio of the concentration of Trp to the concentration of PCae C34:2; and/or
(g) the concentration of SMOH C22:2, the ratio of the concentration of PCaa C36:2 to the concentration of PCae C36:2 and the ratio of the concentration of Trp to the concentration of PCae C34:0; and, optionally, preparing a hard or soft copy comprising the concentration values determined; and
(ii) comparing the values determined in (i) with values obtained from healthy subjects;
wherein an increase in the concentration of the single metabolites in combination with a decrease in the ratio(s) as compared to values obtained from healthy subjects is indicative of endometriosis.

2. The method according to claim 1, further comprising normalising the obtained values.

3. The method according to claim 2, wherein the normalisation is an adjustment for age and/or body mass index.

4. The method according to claim 1, wherein the concentrations are determined by mass spectrometry.

5. The method according to claim 4, wherein the mass spectrometry is selected from liquid chromatography mass spectrometry (LC-MS or HPLC-MS) and tandem mass spectrometry (MS-MS).

6. The method according to claim 1, wherein the concentrations are determined by reference to internal metabolite standards.

7. The method according to claim 6, wherein the internal metabolite standards are stable isotope-labelled metabolite standards.

8. The method according to claim 1, wherein the sample is selected from blood, serum, plasma, saliva, urine, cerebrospinal fluid, condensates from respiratory air, tears, mucosal tissue, mucus, vaginal tissue, endometrium, eutopic endometrium, skin, hair or hair follicle.

9. The method according to claim 1, wherein the subject is a human subject, preferably of Caucasian race.

10. A kit comprising or consisting of
(a) stable isotope-labelled SMOH C16:1 or SMOH of a different mass and/or different a side chain desaturation level or a different desaturation position or a chemically similar compound not naturally occurring in the human sample;
stable isotope-labelled PCaa C36:2 or PCaa of a different mass and/or a different side chain desaturation level or a different desaturation position or a chemically similar compound not naturally occurring in the human sample; and stable isotope-labelled PCae C34:2 or PCae of a different mass and/or a different side chain desaturation level or a different desaturation position or a chemically similar compound not naturally occurring in the human sample;
(b) stable isotope-labelled PCae C30.0 or PCae of a different mass and/or a different side chain desaturation level or a different desaturation position or a chemically similar compound not naturally occurring in the human sample;
stable isotope-labelled PCaa C36.2 or PCaa of a different mass and/or a different side chain desaturation level or a different desaturation position or a chemically similar compound not naturally occurring in the human sample;
stable isotope-labelled PCae C36.2 or PCae of a different mass and/or a different side chain desaturation level or a different desaturation position or a chemically similar compound not naturally occurring in the human sample;
stable isotope-labelled Trp or a chemically similar compound not naturally occurring in the human sample; and
stable isotope-labelled PCae C34.0 or PCae of a different mass and/or a different side chain desaturation level or a different desaturation position or a chemically similar compound not naturally occurring in the human sample;
(c) stable isotope-labelled PCae C36.1 or PCae of a different mass and/or a different side chain desaturation level or a different desaturation position or a chemically similar compound not naturally occurring in the human sample;
stable isotope-labelled PCaa C36.2 or PCaa of a different mass and/or a different side chain desaturation level or a different desaturation position or a chemically similar compound not naturally occurring in the human sample;
stable isotope-labelled PCae C36.2 or PCae of a different mass and/or a different side chain desaturation level or a different desaturation position or a chemically similar compound not naturally occurring in the human sample;
stable isotope-labelled Trp or a chemically similar compound not naturally occurring in the human sample; and
stable isotope-labelled PCae C34.0 or PCae of a different mass and/or a different side chain desaturation level or a different desaturation position or a chemically similar compound not naturally occurring in the human sample;
(d) stable isotope-labelled SM C16.1 or SM of a different mass and/or different a side chain desaturation level or a different desaturation position or a chemically similar compound not naturally occurring in the human sample;
stable isotope-labelled PCaa C36.2 or PCaa of a different mass and/or a different side chain desaturation level or a different desaturation position or a chemically similar compound not naturally occurring in the human sample;
stable isotope-labelled PCae C36.2 or PCae of a different mass and/or a different side chain desaturation level or a different desaturation position or a chemically similar compound not naturally occurring in the human sample;
stable isotope-labelled Trp or a chemically similar compound not naturally occurring in the human sample; and
stable isotope-labelled PCae C34.0 or PCae of a different mass and/or a different side chain desaturation level or a different desaturation position or a chemically similar compound not naturally occurring in the human sample;
(e) stable isotope-labelled SM C16.1 or SM of a different mass and/or different a side chain desaturation level or a different desaturation position or a chemically similar compound not naturally occurring in the human sample;
stable isotope-labelled PCaa C36.2 or PCaa of a different mass and/or a different side chain desaturation level or a different desaturation position or a chemically similar compound not naturally occurring in the human sample;
stable isotope-labelled PCae C36.2 or PCae of a different mass and/or a different side chain desaturation level or a different desaturation position or a chemically similar compound not naturally occurring in the human sample;
stable isotope-labelled Arg or a chemically similar compound not naturally occurring in the human sample; and
stable isotope-labelled PCae C34.2 or PCae of a different mass and/or a different side chain desaturation level or a different desaturation position or a chemically similar compound not naturally occurring in the human sample;
(f) stable isotope-labelled SM C16:1 or SM of a different mass and/or different a side chain desaturation level or a different desaturation position or a chemically similar compound not naturally occurring in the human sample;
stable isotope-labelled PCaa C36:2 or PCaa of a different mass and/or a different side chain desaturation level or a different desaturation position or a chemically similar compound not naturally occurring in the human sample;
stable isotope-labelled PCae C36:2 or PCae of a different mass and/or a different side chain desaturation level or a different desaturation position or a chemically similar compound not naturally occurring in the human sample;
stable isotope-labelled Trp or a chemically similar compound not naturally occurring in the human sample; and
stable isotope-labelled PCae C34:2 or PCae of a different mass and/or a different side chain desaturation level or a different desaturation position or a chemically similar compound not naturally occurring in the human sample; and/or
(g) stable isotope-labelled SMOH C22:2 or SMOH of a different mass and/or different a side chain desaturation level or a different desaturation position or a chemically similar compound not naturally occurring in the human sample;
stable isotope-labelled PCaa C36:2 or PCaa of a different mass and/or a different side chain desaturation level or a different desaturation position or a chemically similar compound not naturally occurring in the human sample;
stable isotope-labelled PCae C36:2 or PCae of a different mass and/or a different side chain desaturation level or a different desaturation position or a chemically similar compound not naturally occurring in the human sample;
stable isotope-labelled Trp or a chemically similar compound not naturally occurring in the human sample; and
stable isotope-labelled PCae C34:0 or PCae of a different mass and/or a different side chain desaturation level or a different desaturation position or a chemically similar compound not naturally occurring in the human sample.

11. The kit according to claim 10, wherein the isotope is selected from the group consisting of an isotope of ¹²C, ¹³C, ¹⁴N, ⁵N and ²H.

12. The kit according to claim 10, further comprising preservatives or buffers for storage.

13. (canceled)

14. The kit according to claim 11, further comprising preservatives or buffers for storage.
